(19) United States
(12) Patent Application Publication
(10) Pub. No.: US 2025/0065597 A1
(43) Pub. Date: Feb. 27, 2025

(54) TEXTILE AND POLYMER LAMINATE LAYERS FOR IMPLANTS

(71) Applicant: EDWARDS LIFESCIENCES CORPORATION, Irvine, CA (US)

(72) Inventors: Radhika Vaid, Irvine, CA (US); Sandip Vasant Pawar, Irvine, CA (US); Rupesh Gajanan Nawalakhe, Trabuco Canyon, CA (US); Gillian Irene Armstrong, Huntington Beach, CA (US); Ido Halabi, Givatayim (IL); Carmel Peleg, Pardes Hana-Karkur (IL); Anna Khodos, Harish (IL); Yaron Herman, Givat Ada (IL)

(21) Appl. No.: 18/945,342

(22) Filed: Nov. 12, 2024

Related U.S. Application Data
(63) Continuation of application No. PCT/US2023/021399, filed on May 8, 2023.
(60) Provisional application No. 63/341,354, filed on May 12, 2022.

(51) Int. Cl. B32B 5/24; B32B 5/02

(52) U.S. Cl. CPC B32B 5/24; B32B 5/024; B32B 2250/40; B32B 2323/043; B32B 2535/00

(57) ABSTRACT

A method for use in the manufacture of a medical implant is provided. A sandwich is formed, the sandwich including a fabric layer; a coating layer comprising a polymer configured to inhibit tissue growth thereon; a binding layer sandwiched between the fabric layer and the coating layer; and at least part of a frame sandwiched between the fabric layer and the binding layer. The sandwich is heated and compressed such that the binding layer binds the fabric layer to the coating layer. Holes are then formed through the sandwich. Other implementations are also described.

### CROSS-REFERENCES TO RELATED APPLICATIONS

This application is a continuation of International Patent Application No. PCT/US2023/021399, filed May 8, 2023, which claims the benefit of U.S. Patent Application No. 63/341,354, filed May 12, 2022, the entire disclosures all of which are incorporated by reference for all purposes.

This application is related to International Patent Application No. PCT/IB2021/060436, filed Nov. 11, 2021; International Patent Application No. PCT/US2021/039587, filed Jun. 29, 2021; and International Patent Application No. PCT/US2022/052834, filed Dec. 14, 2022, the entire disclosures all of which are incorporated by reference for all purposes.

### BACKGROUND

After implantation of an implant, tissue may grow on the implant. In some cases, such tissue growth is desirable, e.g., to help the implant merge with the tissue or organ. For example, in some hernia procedures, a mesh is introduced and anchored around an opening so as to prevent organs from undesirably passing through the opening. The mesh encourages tissue to grow thereon so as to “patch up” the opening. Conversely, in some cases tissue growth on an implant is undesirable. For example, an implant (or component thereof) that is intended to move (e.g., oscillate) within the body may be negatively impacted by tissue growth.

Mechanical characteristics of an implant, such as strength and/or flexibility, typically require that the implant be formed from particular materials. However, promotion or inhibition of tissue growth on an implant may require the use of other materials, and these other materials may not provide the desired mechanical characteristics. Therefore, it can be advantageous to combine materials that provide desirable mechanical characteristics with those that provide desirable tissue growth characteristics.

### SUMMARY

In some implementations, systems, apparatuses, and/or devices such as implants, prosthetic valves, stents and/or patches that are used intracardially and/or against a tissue of a subject, may benefit from at least one tissue-growth inhibiting surface. For example, an implant may benefit from such tissue-growth inhibition at a surface that is in intermittent, recurring, or continuous contact with tissue, and/or at a surface that rarely or never contacts tissue. Described herein are sheets that provide at least one such tissue-growth inhibiting surface, and that can be included in such systems, apparatuses, and/or devices. Methods of forming such sheets are also described. Additionally described are methods for forming implants that include such sheets.

In some implementations, such sheets have a multi-layered structure. In general, the sheets are formed by heating and/or applying pressure to this multi-layered structured, which can comprise a fabric layer and at least one coating and binding layers, e.g., a sandwich. Each binding layer, configured to bind a coating layer to the fabric layer, is sandwiched between the fabric layer and the coating layer. The coating layer can comprise (e.g., can be a film of) a tissue-growth inhibiting material (e.g., polymer). In some implementations, a frame can be introduced into the sandwich, thereby forming a part of the sheet. In some implementations, the sheet can be attached to a frame subsequently to formation of the sheet, e.g., by using stiches, glue and/or applying heat and/or pressure. In some implementations, the sheet can be attached to a frame by folding an edge of the sheet over an edge of the frame to create a hem.

Described herein are implants that incorporate such a sheet. The systems, apparatuses, methods, devices, etc. can be configured to inhibit tissue growth thereon. Various examples of systems, apparatuses, and/or methods for coating an implant inhibiting tissue growth thereon are described. In some implementations, an implant that incorporates the sheet is configured to support, and/or to guide movement of a leaflet of a valve of a heart of a subject. An example of where such an implant can be helpful is when used at the posterior leaflet of a mitral valve experiencing flail, prolapse, rigidity, and/or another issue resulting in regurgitations.

Various implementations of systems, devices, examples of prosthetic implants, etc. can be coated using the coating methods disclosed herein, and any combination of the described features, components, and options can be made unless specifically excluded. For example, various polymers inhibiting tissue growth thereon can be used with any appropriate methods for binding them to an apparatus, implant, etc., even if a specific combination of heat and/or pressure is not explicitly described. Likewise, the different constructions and features of devices can be mixed and matched, such as by combining any sheet with any form of frame, even if not explicitly disclosed. In short, individual components of the disclosed systems can be combined unless mutually exclusive or physically impossible.

Examples herein are directed to towards systems, apparatuses, devices, methods, etc. for forming a sheet having at least one tissue inhibiting growth surface and/or for coating a frame thereby providing it with at least one tissue inhibiting growth surface. In some implementations the sheet alone and/or in combination with the frame can be used to mitigate leaflet flail, prolapse, abnormal leaflet motion, and/or other problems.

There is provided, in accordance with some implementations, a method, including forming a sandwich including a binding layer sandwiched between (i) a fabric layer, and (ii) a coating layer that includes a polymer configured to inhibit tissue growth thereon. The method can further include forming a sheet by binding the fabric layer to the coating layer by heating and/or compressing the sandwich.

In some implementations: (i) in the sandwich, the binding layer is unbound from the fabric layer, (ii) forming the sandwich includes forming the sandwich in which the binding layer is unbound from the fabric layer, and/or (iii) forming the sheet by binding the fabric layer to the coating layer includes forming the sheet by binding the binding layer to the fabric layer and to the coating layer by heating and compressing the sandwich.

In some implementations: (i) in the sandwich, the binding layer is bound to the fabric layer, (ii) forming the sandwich includes forming the sandwich in which the binding layer is bound to the fabric layer, and/or (iii) forming the sheet by binding the fabric layer to the coating layer includes forming the sheet by binding, to the coating layer, the binding layer that is bound to the fabric layer, by heating and compressing the sandwich.

In some implementations, the coating layer consists essentially of the polymer.

In some implementations, the coating layer is a film.

In some implementations, the coating layer is nonporous.

In some implementations, the coating layer is porous.

In some implementations, the fabric is a woven fabric.

In some implementations, the fabric is a nonwoven fabric.

In some implementations, the polymer is an ultra-high-molecular-weight polyethylene (UHMWPE).

In some implementations, the polymer is a fluorinated ethylene propylene (FEP).

In some implementations, the binding layer includes an acrylate.

In some implementations, the binding layer is a siloxane.

In some implementations, the binding layer includes a thermoplastic polyurethane (TPU).

In some implementations, the fabric layer is 20-150 microns thick.

In some implementations, the binding layer is 5-30 microns thick.

In some implementations: (A) the coating layer defines a first face of the sheet, and the sheet has a second face opposite to the first face; (B) forming the sandwich includes forming the sandwich to define a lip in which a lip-portion of the coating layer and a lip-portion of the binding layer extend together beyond an edge of the fabric layer, (C) the method further includes folding the lip around the edge of the fabric layer onto the second face, such that the lip-portion of the binding layer is sandwiched between the lip-portion of the coating layer and the second face; and/or (D) forming the sheet includes forming the sheet by: (i) heating the sandwich including the lip folded around the edge of the fabric layer onto the second face, and (ii) compressing the sandwich including the lip folded around the edge of the fabric layer onto the second face.

In some implementations, forming the sheet includes forming the sheet by binding the binding layer to the fabric layer by heating and compressing the binding layer against the fabric layer.

In some implementations, forming the sheet includes forming the sheet by binding the coating layer to the binding layer by heating and compressing the coating layer against the binding layer.

In some implementations, forming the sandwich includes defining a lip in which a lip-portion of the coating layer and a lip-portion of the binding layer extend together beyond an edge of the fabric layer.

In some implementations, forming the sandwich includes: (i) forming the sandwich such that the coating layer defines a first face of the sheet, and such that the sheet has a second face opposite to the first face, and (ii) folding the lip onto the second face such that the lip-portion of the binding layer is sandwiched between the lip-portion of the coating layer and the second face.

In some implementations: (A) the sandwich includes at least part of a frame sandwiched between the fabric layer and the binding layer, and (B) forming the sheet includes forming the sheet by binding the fabric layer to the coating layer, with at least the part of the frame disposed between the fabric layer and the binding layer, by: (i) heating the sandwich that includes at least the part of the frame sandwiched between the fabric layer and the binding layer, and (i) compressing the sandwich that includes at least the part of the frame sandwiched between the fabric layer and the binding layer.

In some implementations, forming the sheet includes forming the sheet to define a pocket between the fabric layer and the binding layer by heating and compressing the sandwich around a region of the pocket, such that at least part of the frame is disposed in the pocket.

In some implementations, heating the sandwich includes heating the sandwich between plates, at least one of the plates having a recess at the region of the pocket.

In some implementations, compressing the sandwich includes compressing the sandwich between plates, at least one of the plates having a recess at the region of the pocket.

In some implementations, forming the sheet includes forming the sheet by binding the fabric layer to the coating layer in a manner that forms a pocket between the fabric layer and the binding layer.

In some implementations, forming the pocket by: (i) heating the sandwich around an area of the pocket, and (ii) compressing the sandwich around the area of the pocket.

In some implementations, the heating the sandwich around a circumscribed area of the sandwich.

In some implementations, the compressing of the sandwich around a circumscribed area of the sandwich.

In some implementations, the method further includes closing the pocket by binding the fabric layer and the first binding layer circumscribing the pocket.

In some implementations, forming the sheet includes forming the sheet having an opening at an edge of the sheet by having the fabric layer unbound from the first binding layer.

In some implementations, forming the sandwich includes forming the sandwich having the frame is sandwiched, along with the binding layer, between the fabric layer and the coating layer.

In some implementations, a thickness of the frame between the fabric layer and the binding layer is 20-500 microns.

In some implementations, the method further includes covering at least part of a frame by fixedly attaching the sheet to the frame.

In some implementations, forming the sandwich includes forming the sandwich including the frame sandwiched, along with the binding layer, between the fabric layer and the coating layer.

In some implementations, a thickness of the frame between the fabric layer and the binding layer is 20-500 microns.

In some implementations, fixedly attaching the sheet to the frame includes stitching the sheet to the frame.

In some implementations, the frame has a first side and an opposite second side, and wrapping the fabric layer around the frame from the first side to the second side, such that at least part of the frame is sandwiched between (i) a first part of the fabric layer disposed against the first side of the frame, and (ii) a second part of the fabric layer disposed against the second side of the frame.

In some implementations, the method further comprises forming multiple holes through the sheet, to facilitate blood flow therethrough.

In some implementations, the method further comprises flattening the sheet and keeping it flat while forming the multiple holes through the sheet.

In some implementations, forming the holes includes forming the holes by laser cutting.

In some implementations, each of the holes is hexagonal.

In some implementations, forming the sheet includes forming the sheet having a pocket between the fabric layer and the binding layer by heating and compressing the sandwich around an area defining the pocket, such that at least part of the frame being disposed in the pocket.

In some implementations, heating the sandwich includes positioning the sandwich between plates, at least one of the plates is heated, and at least one of the plates having a recess so as forming the pocket at the recess.

In some implementations, compressing the sandwich includes compressing the sandwich between plates, at least one of the plates having a recess so as forming the pocket at the recess.

In some implementations, (A) forming the sandwich includes forming the second sandwich including a second binding layer sandwiched between: (i) the fabric layer, and (ii) a second coating layer configured to inhibit tissue growth thereon; and (B) forming the sheet by binding the fabric layer to the second coating layer by: (i) heating the sandwich, and (ii) compressing the sandwich.

In some implementations, the coating layer defines a first face of the sheet, and the sheet has a second face opposite to the first face. In some implementations, forming the sandwich includes forming the sandwich to define a lip-portion of the coating layer and a lip-portion of the binding layer extend together beyond an edge of the fabric layer.

In some implementations, the method further includes folding the lip around the edge of the fabric layer onto the second face, such that the lip-portion of the binding layer is sandwiched between the lip-portion of the coating layer and the second face.

In some implementations, forming the sheet includes forming the sheet by: (i) heating the sandwich including the lip folded around the edge of the fabric layer onto the second face, and (ii) compressing the sandwich including the lip folded around the edge of the fabric layer onto the second face.

In some implementations, the first coating layer, the second coating layer, and the first binding layer extend together beyond an edge of the fabric layer, thereby defining a lip, and forming the sandwich including the lip in which a lip-portion of the first coating layer, a lip-portion of the second coating layer and a lip-portion of the first binding layer are disposed.

In some implementations, forming the sandwich includes: (i) forming the sandwich such that the coating layer defines a first face of the sheet, and such that the sheet has a second face opposite to the first face, and (ii) folding the lip onto the second face such that the lip-portion of the binding layer is sandwiched between the lip-portion of the coating layer and the second face.

In some implementations, the first coating layer, the second coating layer, the first binding layer and the second binding layer extend together beyond an edge of the fabric layer, thereby defining a lip, and forming the sandwich including the lip in which a lip-portion of the first coating layer, a lip-portion of the second coating layer and a lip-portion of the first binding layer are disposed.

In some implementations, forming the sheet includes forming the sheet by binding the fabric layer to the coating layer in a manner that forms a pocket between the fabric layer and the binding layer.

In some implementations, forming the sheet in the manner that forms the pocket includes: (i) heating the sandwich around an area of the pocket, and (ii) compressing the sandwich around the area of the pocket.

In some implementations, the heating or compressing of the sandwich is around a circumscribed area of the sandwich.

In some implementations, the method further includes closing the pocket by binding between the fabric layer and the first binding layer circumscribing the pocket.

In some implementations, the sheet defines an opening into the pocket.

In some implementations, at the opening, the fabric layer is unbound from the first binding layer.

In some implementations, the opening is disposed at an edge the sheet.

In some implementations, the sheet further includes a cord at least a portion of the cord extending through the pocket.

In some implementations, the cord is slidable within the pocket.

In some implementations, the polymer is a polytetrafluoroethylene (PTFE).

In some implementations, the polymer is an expanded PTFE (ePTFE).

In some implementations, the coating layer includes several layered sublayers of the polymer.

In some implementations, each of the sublayers is anisotropic, and the sublayers are arranged such that each of the sublayers is disposed orthogonally to an adjacent one of the sublayers.

In some implementations, the coating layer includes less than ten of the sublayers.

In some implementations, the coating layer includes less than six of the sublayers.

In some implementations, compressing the sandwich includes compressing the sandwich at a pressure of 0.01-1 MPa.

In some implementations, compressing the sandwich includes holding the sandwich at the pressure for 100-500 seconds.

In some implementations, heating the sandwich includes heating the sandwich to a temperature of 37-260 degrees Celsius.

In some implementations, heating the sandwich includes holding the sandwich at the temperature for 100-500 seconds.

There is provided, in accordance with some implementations, a system and/or an apparatus, including a sheet that includes: (i) a fabric layer; (ii) a coating layer including a polymer configured to inhibit tissue growth thereon; and/or (iii) a binding layer sandwiched between the fabric layer and the coating layer, and binding the fabric layer to the coating layer.

In some implementations, the system/apparatus is shaped to define a leaflet-augmentation patch, configured to be attached to a leaflet of a valve of a heart of a subject.

In some implementations, the coating layer consists essentially of the polymer.

In some implementations, the coating layer is a film.

In some implementations, the coating layer is nonporous.

In some implementations, the coating layer is porous.

In some implementations, the fabric is a woven fabric.

In some implementations, the fabric is a nonwoven fabric.

In some implementations, the polymer is an ultra-high-molecular-weight polyethylene (UHMWPE).

In some implementations, the polymer is a fluorinated ethylene propylene (FEP).

In some implementations, the binding layer includes an acrylate.

In some implementations, the binding layer is a siloxane.

In some implementations, the fabric layer is 20-150 microns thick.

In some implementations, the binding layer is 5-30 microns thick.

In some implementations, the coating layer and the binding layer extend together beyond an edge of the fabric layer, thereby defining a lip in which a lip-portion of the coating layer and a lip-portion of the binding layer are disposed.

In some implementations, the coating layer defines a first face of the sheet, and the sheet has a second face opposite to the first face, and the lip is folded onto the second face, such that the lip-portion of the binding layer is sandwiched between the lip-portion of the coating layer and the second face.

In some implementations, the lip is folded around the edge of the fabric layer, thereby defining a hem of the sheet within which the fabric layer is disposed between the lip-portion of the binding layer and another portion of the binding layer.

In some implementations, the sheet defines a pocket between the fabric layer and the binding layer.

In some implementations, the binding between the fabric layer and the binding layer circumscribes the pocket, thereby closing the pocket.

In some implementations, the sheet defines an opening into the pocket.

In some implementations, at the opening, the fabric layer is unbound from the first binding layer.

In some implementations, the opening is disposed at an edge of the sheet.

In some implementations, the system/apparatus further includes a cord, at least a portion of the cord extending through the pocket.

In some implementations, the portion of the cord is slidable within the pocket.

In some implementations, the system/apparatus is shaped as a wing defining a contact face, and an opposing face opposite to the contact face.

In some implementations, the wing has multiple holes configured to facilitate blood flow therethrough.

In some implementations, each of the holes is hexagonal.

In some implementations, the wing configured to be anchored to a tissue of a heart of a subject having a first leaflet and a second opposing leaflet, such that the wing extends over the first leaflet toward the second leaflet, and the contact face faces the first leaflet.

In some implementations, the apparatus further includes a frame, the sheet being fixedly attached to the frame in a manner that covers at least part of the frame.

In some implementations, the sheet is fixedly attached to the frame by stitching.

In some implementations, the sheet is fixedly attached to the frame by adhesive.

In some implementations, the sheet is fixedly attached to the frame by the frame being sandwiched between the fabric layer and the binding layer.

In some implementations: (i) the coating layer is a first coating layer, (ii) the binding layer is a first binding layer, and (iii) the sheet further includes: a second coating layer, and a second binding layer, sandwiched between the frame and the second coating layer.

In some implementations, a thickness of the frame between the fabric layer and the binding layer is 20-500 microns.

In some implementations, the frame has a first side and an opposite second side, and the fabric layer is wrapped around from the first side to the second side, such that at least part of the frame is sandwiched between (i) a first part of the fabric layer disposed against the first side of the frame, and (ii) a second part of the fabric layer disposed against the second side of the frame.

In some implementations: (A) the coating layer is a first coating layer, (B) the binding layer is a first binding layer, and (C) the sheet further includes: (i) a second coating layer, and (ii) a second binding layer, sandwiched between the second part of the fabric layer and the second coating layer.

In some implementations, the frame is flexible, and the system/apparatus defines a wing having a contact face, and an opposing face opposite to the contact face.

In some implementations, the sheet has multiple holes configured to facilitate blood flow therethrough.

In some implementations, the wing is configured to be anchored to an annulus of a valve of a heart of a subject such that the wing extends over a first leaflet of the valve toward an opposing leaflet of the valve, and the contact face faces the first leaflet.

In some implementations, the system/apparatus has a pocket between the fabric layer and the binding layer, at least part of the frame being disposed in the pocket.

In some implementations, the coating layer and the binding layer extend together beyond an edge of the frame, thereby defining a lip in which a lip-portion of the coating layer and a lip-portion of the binding layer are disposed.

In some implementations, the frame is disposed between the fabric layer and the binding layer, and the fabric layer extends, together with the coating layer and the binding layer, beyond the edge of the frame.

In some implementations, the coating layer and the binding layer extend together beyond an edge of the fabric layer, thereby defining the lip.

In some implementations, the coating layer defines a first face of the sheet, and the sheet has a second face opposite to the first face, and the lip is folded onto the second face, such that the lip-portion of the binding layer is disposed between the lip-portion of the coating layer and the second face.

In some implementations, at least part of the frame is disposed between the lip-portion of the binding layer and another portion of the binding layer.

In some implementations, the lip is folded around the edge of the frame, thereby defining a hem of the sheet within which the edge of the frame is disposed between the lip-portion of the binding layer and another portion of the binding layer.

In some implementations, at least a part of the fabric layer is disposed within the hem.

In some implementations, the coating layer is a first coating layer, the binding layer is a first binding layer, and the sheet further includes: (i) a second coating layer; and (ii) a second binding layer, sandwiched between the fabric layer and the second coating layer, and binding the fabric layer to the second coating layer, the fabric layer sandwiched between the first binding layer and the second binding layer.

In some implementations, the sheet defines a pocket between the fabric layer and the first binding layer.

In some implementations, the first coating layer, the second coating layer, and the first binding layer extend together beyond an edge of the fabric layer, thereby defining a lip in which a lip-portion of the first coating layer, a lip-portion of the second coating layer, and a lip-portion of the first binding layer are disposed.

In some implementations, within the lip, the lip-portion of the first coating layer is bound to the lip-portion of the second coating layer by the lip-portion of the first binding layer.

In some implementations, a lip-portion of the second binding layer extends beyond the edge of the fabric layer into the lip such that, within the lip, the lip-portion of the first coating layer is bound to the lip-portion of the second coating layer by the lip-portion of the first binding layer and the lip-portion of the second binding layer.

In some implementations, the system/apparatus further includes a frame, disposed between the first and the second binding layers.

In some implementations, at at least part of the sheet, the lip extends beyond an edge of the frame.

In some implementations, at least part of the frame extends beyond an edge of the lip.

In some implementations, the first coating layer and the first binding layer extend together beyond an edge of the fabric layer, thereby defining a lip in which a lip-portion of the first coating layer and a lip-portion of the first binding layer are disposed.

In some implementations, the first coating layer defines a first face of the sheet, and the sheet has a second face opposite to the first face, and the lip is folded onto the second face such that the lip-portion of the first binding layer is disposed between the lip-portion of the first coating layer and the second face.

In some implementations, the lip is folded onto the second face such that the lip-portion of the first binding layer is disposed between the lip-portion of the first coating layer and the second coating layer.

In some implementations, the system/apparatus further includes a frame, the sheet being fixedly attached to the frame in a manner that covers at least part of the frame, and: (i) the first coating layer defines a first face of the sheet, and the sheet has a second face opposite to the first face, and (ii) a lip-portion of the first coating layer and a lip-portion of the first binding layer extend together beyond an edge of the fabric layer, thereby defining a lip, the lip extending beyond an edge of the frame.

In some implementations, the lip is folded onto the second face, thereby defining a hem of the sheet within which at least a part of the frame is disposed.

In some implementations, a lip-portion of the second coating layer and a lip-portion of the second binding layer extend together beyond an edge of the fabric, into the lip.

In some implementations, the system/apparatus further includes a frame, the sheet being fixedly attached to the frame in a manner that covers at least part of the frame.

In some implementations, the system/apparatus has a pocket disposed between the fabric layer and the first binding layer, at least part of the frame being disposed in the pocket, between the fabric layer and the first binding layer.

In some implementations, the pocket is closed by the binding between the fabric layer and the first binding layer circumscribing the pocket.

In some implementations, the sheet defines an opening into the pocket.

In some implementations, at the opening, the fabric layer is unbound from the first binding layer.

In some implementations, the opening is disposed at an edge of the sheet.

In some implementations, the system/apparatus further includes a cord, attached to the frame, at least a portion of the cord extending through the pocket.

In some implementations, the portion of the cord is slidable within the pocket.

In some implementations, the polymer is a polytetrafluoroethylene (PTFE).

In some implementations, the polymer is an expanded PTFE (ePTFE).

In some implementations, the coating layer includes several layered sublayers of the polymer.

In some implementations, each of the sublayers is anisotropic, and the sublayers are arranged such that each of the sublayers is disposed orthogonally to an adjacent one of the sublayers.

In some implementations, the coating layer includes less than ten of the sublayers.

In some implementations, the coating layer includes less than six of the sublayers.

In some implementations, the binding layer includes a thermoplastic polyurethane (TPU).

In some implementations, the TPU is a polycarbonate-based TPU.

In some implementations, the polycarbonate-based TPU is a polycarbonate-based aliphatic thermoplastic polyurethane.

In some implementations, the sheet is formed by binding the binding layer to the fabric layer and the coating layer by compressing together and heating the fabric layer, the coating layer, and the binding layer.

In some implementations, the sheet is formed by compressing together the fabric layer, the coating layer, and the binding layer for 100-500 seconds.

In some implementations, the sheet is formed by compressing together the fabric layer, the coating layer, and the binding layer for 20-420 seconds.

In some implementations, the sheet is formed by heating the fabric layer, the coating layer, and the binding layer for 100-500 seconds.

In some implementations, the sheet is formed by heating the fabric layer, the coating layer, and the binding layer for 20-420 seconds.

In some implementations, the sheet is formed by compressing together the fabric layer, the coating layer, and the binding layer at a pressure of 0.01-1 MPa.

In some implementations, the sheet is formed by compressing together the fabric layer, the coating layer, and the binding layer at the pressure for 100-500 seconds.

In some implementations, the sheet is formed by compressing together the fabric layer, the coating layer, and the binding layer at the pressure for 20-420 seconds.

In some implementations, the sheet is formed by heating the fabric layer, the coating layer, and the binding layer to a temperature of 37-260 degrees Celsius.

In some implementations, the sheet is formed by holding the fabric layer, the coating layer, and the binding layer at the temperature for 100-500 seconds.

In some implementations, the sheet is formed by holding the fabric layer, the coating layer, and the binding layer at the temperature for 20-420 seconds.

There is provided, in accordance with some implementations, a system and/or an apparatus, including a cardiovascular implant that includes a frame and/or a sheet. The sheet can include a fabric layer secured against at least a portion of the frame. The sheet can further include a coating layer that includes a polymer configured to inhibit tissue growth thereon. The sheet can further include a binding layer, sandwiched between the fabric layer and the coating layer, and binding the fabric layer to the coating layer.

In some implementations, the portion of the frame is a first portion of the frame, and a second portion of the frame is exposed from the sheet.

In some implementations, the frame is designed as a frame for a prosthetic valve.

In some implementations, the frame is designed as a frame for a stent.

In some implementations, the frame is designed as a leaflet-augmentation patch.

In some implementations, the frame is designed as a wing.

In some implementations, the implant is configured to be anchored to a tissue of a heart the subject.

In some implementations, the sheet is fixedly attached to the frame by stitching.

In some implementations, the sheet is fixedly attached to the frame by adhesive.

In some implementations, the system/apparatus has a pocket between the fabric layer and the binding layer, at least part of the frame being disposed in the pocket.

In some implementations, the coating layer consists essentially of the polymer.

In some implementations, the coating layer is a film.

In some implementations, the coating layer is nonporous.

In some implementations, the coating layer is porous.

In some implementations, the fabric is a woven fabric.

In some implementations, the fabric is a nonwoven fabric.

In some implementations, the polymer is an ultra-high-molecular-weight polyethylene (UHMWPE).

In some implementations, the polymer is a fluorinated ethylene propylene (FEP).

In some implementations, the binding layer includes an acrylate.

In some implementations, the binding layer is a siloxane.

In some implementations, the fabric layer is 20-150 microns thick.

In some implementations, the binding layer is 5-30 microns thick.

In some implementations, the coating layer and the binding layer extend together beyond an edge of the frame, thereby defining a lip in which a lip-portion of the coating layer and a lip-portion of the binding layer are disposed.

In some implementations, the frame is disposed between the fabric layer and the binding layer, and the fabric layer extends, together with the coating layer and the binding layer, beyond the edge of the frame.

In some implementations, the coating layer and the binding layer extend together beyond an edge of the fabric layer, thereby defining the lip.

In some implementations, the coating layer defines a first face of the sheet, and the sheet has a second face opposite to the first face, and the lip is folded onto the second face, such that the lip-portion of the binding layer is disposed between the lip-portion of the coating layer and the second face.

In some implementations, at least part of the frame is disposed between the lip-portion of the binding layer and another portion of the binding layer.

In some implementations, the lip is folded around the edge of the frame, thereby defining a hem of the sheet within which the edge of the frame is disposed between the lip-portion of the binding layer and another portion of the binding layer.

In some implementations, at least a part of the fabric layer is disposed within the hem.

In some implementations, the fabric layer is secured against at least the portion of the frame by at least the portion of the frame being sandwiched between the binding layer and the fabric layer.

In some implementations, the portion of the frame is a first portion of the frame, and a second portion of the frame is disposed within a pocket between the binding layer and the fabric layer.

In some implementations, the sheet is fixedly attached to the frame by the frame being sandwiched between the fabric layer and the binding layer.

In some implementations, a thickness of the frame between the fabric layer and the binding layer is 20-500 microns.

In some implementations, the frame has a first side and an opposite second side, and the fabric layer is wrapped around from the first side to the second side, such that at least part of the frame is sandwiched between (i) a first part of the fabric layer disposed against the first side of the frame, and (ii) a second part of the fabric layer disposed against the second side of the frame.

In some implementations, the coating layer is a first coating layer, the binding layer is a first binding layer, and the sheet further includes: (i) a second coating layer, and (ii) a second binding layer, sandwiched between the second part of the fabric layer and the second coating layer.

In some implementations, the frame is flexible, and the implant defines a wing having a contact face, and an opposing face opposite to the contact face.

In some implementations, the sheet has multiple holes configured to facilitate blood flow therethrough.

In some implementations, the wing is configured to be anchored to an annulus of a valve of a heart of a subject such that the wing extends over a first leaflet of the valve toward an opposing leaflet of the valve, and the contact face faces the first leaflet.

In some implementations, the coating layer is a first coating layer, the binding layer is a first binding layer, and the sheet further includes: (i) a second coating layer; and (ii) a second binding layer, sandwiched between the fabric layer and the second coating layer, and binding the fabric layer to the second coating layer, the fabric layer sandwiched between the first binding layer and the second binding layer.

In some implementations, the sheet defines a pocket between the fabric layer and the first binding layer.

In some implementations, the first coating layer and the first binding layer extend together beyond an edge of the fabric layer, thereby defining a lip in which a lip-portion of the first coating layer and a lip-portion of the first binding layer are disposed.

In some implementations, the first coating layer defines a first face of the sheet, and the sheet has a second face opposite to the first face, and the lip is folded onto the second face such that the lip-portion of the first binding layer is disposed between the lip-portion of the first coating layer and the second face.

In some implementations, the lip is folded onto the second face such that the lip-portion of the first binding layer is disposed between the lip-portion of the first coating layer and the second coating layer.

In some implementations, the first coating layer, the second coating layer, and the first binding layer extend together beyond an edge of the fabric layer, thereby defining a lip in which a lip-portion of the first coating layer, a lip-portion of the second coating layer, and a lip-portion of the first binding layer are disposed.

In some implementations, within the lip, the lip-portion of the first coating layer is bound to the lip-portion of the second coating layer by the lip-portion of the first binding layer.

In some implementations, a lip-portion of the second binding layer extends beyond the edge of the fabric layer into the lip such that, within the lip, the lip-portion of the first coating layer is bound to the lip-portion of the second coating layer by the lip-portion of the first binding layer and the lip-portion of the second binding layer.

In some implementations, at at least part of the sheet, the lip extends beyond an edge of the frame.

In some implementations, at least part of the frame extends beyond an edge of the lip.

In some implementations, the lip is folded onto the second face, thereby defining a hem of the sheet within which at least a part of the frame is disposed.

In some implementations, a lip-portion of the second coating layer and a lip-portion of the second binding layer extend together beyond an edge of the fabric, into the lip.

In some implementations, the sheet being fixedly attached to the frame in a manner that covers at least part of the frame.

In some implementations, the system/apparatus has a pocket disposed between the fabric layer and the first binding layer, at least part of the frame being disposed in the pocket, between the fabric layer and the first binding layer.

In some implementations, the pocket is closed by the binding between the fabric layer and the first binding layer circumscribing the pocket.

In some implementations, the sheet defines an opening into the pocket.

In some implementations, at the opening, the fabric layer is unbound from the first binding layer.

In some implementations, the opening is disposed at an edge of the sheet.

In some implementations, the system/apparatus further includes a cord, attached to the frame, at least a portion of the cord extending through the pocket.

In some implementations, the portion of the cord is slidable within the pocket.

In some implementations, the polymer is a polytetrafluoroethylene (PTFE).

In some implementations, the polymer is an expanded PTFE (ePTFE).

In some implementations, the coating layer includes several layered sublayers of the polymer.

In some implementations, each of the sublayers is anisotropic, and the sublayers are arranged such that each of the sublayers is disposed orthogonally to an adjacent one of the sublayers.

In some implementations, the coating layer includes less than ten of the sublayers.

In some implementations, the coating layer includes less than six of the sublayers.

In some implementations, the binding layer includes a thermoplastic polyurethane (TPU).

In some implementations, the TPU is a polycarbonate-based TPU.

In some implementations, the polycarbonate-based TPU is a polycarbonate-based aliphatic thermoplastic polyurethane.

In some implementations, the sheet is formed by binding the binding layer to the fabric layer and the coating layer by compressing together and heating the fabric layer, the coating layer, and the binding layer.

In some implementations, the sheet is formed by heating the fabric layer, the coating layer, and the binding layer for 100-500 seconds.

In some implementations, the sheet is formed by heating the fabric layer, the coating layer, and the binding layer for 20-420 seconds.

In some implementations, the sheet is formed by compressing together the fabric layer, the coating layer, and the binding layer for 100-500 seconds.

In some implementations, the sheet is formed by compressing together the fabric layer, the coating layer, and the binding layer for 20-420 seconds.

In some implementations, the sheet is formed by compressing together the fabric layer, the coating layer, and the binding layer at a pressure of 0.01-1 MPa.

In some implementations, the sheet is formed by compressing together the fabric layer, the coating layer, and the binding layer at the pressure for 100-500 seconds.

In some implementations, the sheet is formed by compressing together the fabric layer, the coating layer, and the binding layer at the pressure for 20-420 seconds.

In some implementations, the sheet is formed by heating the fabric layer, the coating layer, and the binding layer to a temperature of 37-260 degrees Celsius.

In some implementations, the sheet is formed by holding the fabric layer, the coating layer, and the binding layer at the temperature for 100-500 seconds.

In some implementations, the sheet is formed by holding the fabric layer, the coating layer, and the binding layer at the temperature for 20-420 seconds.

There is provided, in accordance with some implementations, a method, including temporarily flattening a sheet of an implant by constraining a frame of the implant, the sheet being fixedly attached to the frame. The method can further include forming holes in the sheet while the sheet remains flattened. The method can further include subsequently allowing the frame to unflatten toward a resting shape in which the frame holds the sheet in a curved configuration.

In some implementations, forming the holes includes forming the holes using an automated laser cutting system.

In some implementations, forming the holes includes forming the holes by moving a laser head of a laser cutting system along a plane parallel to the flattened sheet.

In some implementations, flattening the sheet includes flattening the sheet using a cage having rails configured to hold the sheet in the flat configuration.

In some implementations, the method further includes forming the sheet by binding a fabric layer to a coating layer by: (i) forming a sandwich including a binding layer sandwiched between the fabric layer and the coating layer; and (ii) heating and/or compressing the sandwich.

In some implementations, the method further includes, subsequently to forming the sheet, and prior to temporarily flattening the sheet, fixedly attaching the sheet to the frame.

In some implementations: (i) the frame is a first frame of multiple frames, fixedly attached to the sheet, (ii) flattening the sheet by constraining the frame includes flattening the sheet by concurrently constraining the multiple frames while the multiple frames remain fixedly attached to the sheet, and (iii) allowing the frame to unflatten includes allowing the multiple frames to unflatten.

In some implementations, the method further includes attaching the multiple frames in an arrangement across the sheet prior to flattening the sheet.

In some implementations, the method further includes, subsequently to allowing the multiple frames to unflatten, cutting the sheet to separate the multiple frames thereby forming multiple implants.

In some implementations, cutting the sheet includes cutting the sheet while the multiple frames remain constrained.

In some implementations, forming the holes includes forming the holes using an automated cutting device.

The present invention will be more fully understood from the following detailed description of implementations thereof, taken together with the drawings, in which:

### BRIEF DESCRIPTION OF THE DRAWINGS

FIGS. 1A-D are schematic illustrations of a method for forming an example sheet in accordance with some implementations;

FIGS. 2A-C are schematic illustrations of a sheet defining a pocket in accordance with some implementations;

FIG. 3 is a schematic illustration of an example laminating apparatus in accordance with some implementations;

FIGS. 4A-K are schematic illustrations of an example sheet being fixedly attached to a frame in accordance with some implementations;

FIGS. 5A-B are schematic illustrations of an example implant for use with a valve of a heart of a subject, in accordance with some implementations;

FIGS. 6A-D are schematic illustrations of an example implant for use with a valve of a heart of a subject, in accordance with some implementations;

FIGS. 7A-B are schematic illustrations of an example laminating apparatus, in accordance with some implementations; and

FIGS. 8A-C are schematic illustrations of a technique for forming holes through a sheet of an implant, in accordance with some implementations.

### DETAILED DESCRIPTION

Systems, apparatuses, methods, devices, etc. for inhibiting tissue growth on an implantable sheet are described herein. In some implementations, systems, apparatuses, methods, devices, etc. include an apparatus that comprises a sheet constructed of a multi-layered structure and/or implants that incorporate the sheet. The systems, apparatuses, methods, devices, etc. can be configured to inhibit tissue growth thereon. Various examples of apparatuses and/or methods for coating an implant inhibiting tissue growth thereon are described. In some implementations, an implant that incorporates the sheet is configured to support, and/or to guide movement of a leaflet of a valve of a heart of a subject. An example of where such an implant can be helpful is when used at the posterior leaflet of a mitral valve experiencing flail, prolapse, rigidity, and/or another issue resulting in regurgitations.

The described systems, apparatuses, methods, devices, etc. should not be construed as limiting in anyway. Instead, the present disclosure is directed toward all novel and nonobvious features and aspects of the various disclosed coating procedures and implementations, alone and in various combinations and sub-combinations with one another. The disclosed systems, apparatuses, methods, devices, etc. are not limited to any specific aspect, feature, or combination thereof, nor do the disclosed systems, apparatuses, methods, devices, etc. require that any one or more specific advantages be present or problems be solved. Further, techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

Various implementations of systems, devices, examples of prosthetic implants, etc. can be coated using the coating methods disclosed herein, and any combination of the described features, components, and options can be made unless specifically excluded. For example, various polymers inhibiting tissue growth thereon can be used with any appropriate methods for binding them to an apparatus, even if a specific combination of heat and/or pressure is not explicitly described. Likewise, the different constructions and features of devices can be mixed and matched, such as by combining any sheet with any form of frame, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

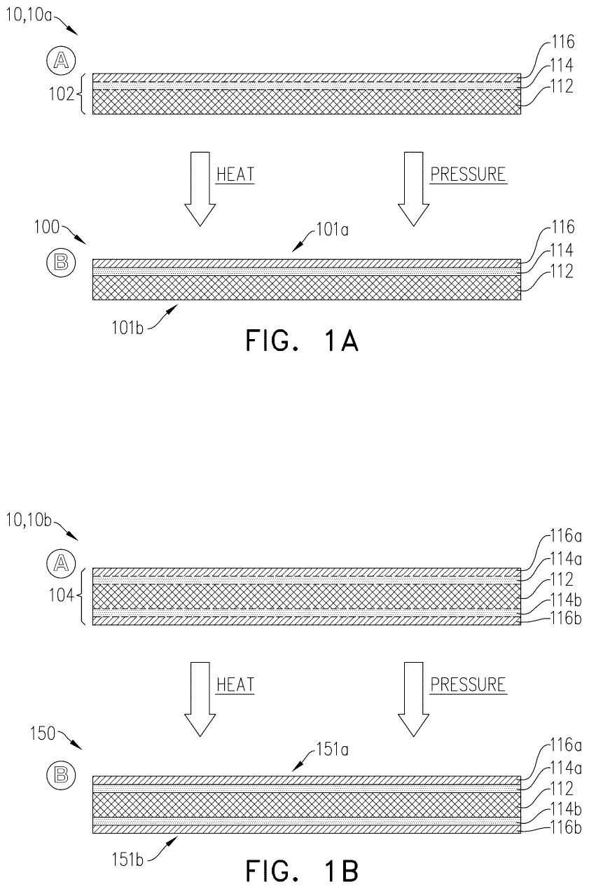

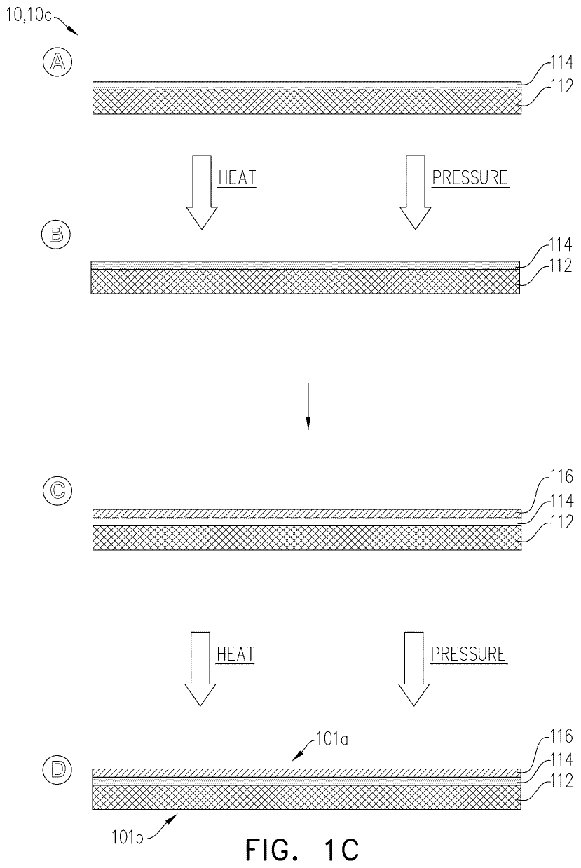

Reference is now made to FIGS. 1A-D, which are schematic illustrations of a method 10, and variants thereof, for forming various sheets, in accordance with some implementations. These sheets are typically implantable in a subject, either alone or as a component of an implant. FIGS. 1A and 1C illustrate methods 10a and 10c for forming a sheet 100, these methods being variants of method 10. FIGS. 1B and 1D illustrate methods 10b and 10d for forming a sheet 150, these methods also being variants of method 10. In the examples shown, sheet 100 has three layers, whereas sheet 150 has five layers.

Sheet 100 comprises a sandwich 102 that comprises a fabric layer 112, a coating layer 116 and a binding layer 114 sandwiched between the fabric layer and the coating layer and binding the fabric layer to the coating layer. Coating layer 116 comprises a polymer configured to inhibit tissue growth thereon, and can function as a first face 101a of sheet 100. In some implementations, sheet 100, and/or an implant comprising sheet 100, can be configured to be placed against a tissue surface of a subject, with first face 101a facing the tissue surface of the subject. In some implementations, sheet 100 and/or an implant comprising sheet 100, can be configured to be placed against a tissue surface of the subject, with first face 101a facing away from the tissue surface, e.g., with a second face 101b (possibly defined by fabric layer 112) facing the tissue surface.

Sheet 150 comprises a sandwich 104 that comprises fabric layer 112, two coating layers 116 (e.g., a first coating layer 116a and a second coating layer 116b), one on either side of the fabric layer, and two binding layers 114 (e.g., a first binding layer 114a and a second binding layer 114b), each sandwiched between a respective one of the coating layers and a respective side of the fabric layer. In some implementations, sheet 150 can be considered to include the three layers of sandwich 102 (detailed hereinabove with respect to FIG. 1A) as well as an additional coating layer 116 and an additional binding layer 114. Since sheet 150 comprises two layers more than sheet 100, it can be at least slightly thicker than sheet 100. Compared with sheet 100, the two coating layers of sheet 150 can provide tissue growth inhibition on both a first face 151a and a second face 151b of the sheet—which may be advantageous in some implementations.

In some implementations, the tissue-growth-inhibiting polymer of coating layer 116 is an ultra-high-molecular-weight polyethylene (UHMWPE). In some implementations, the tissue-growth-inhibiting polymer of coating layer 116 is a fluorinated ethylene propylene (FEP). In some implementations, the tissue-growth-inhibiting polymer of coating layer 116 is a thermoplastic polyurethane (TPU), e.g., a polycarbonate-based TPU and/or an aliphatic TPU. In some implementations, the tissue-growth-inhibiting polymer of coating layer 116 is a polytetrafluoroethylene (PTFE), such as an expanded PTFE (ePTFE).

In some implementations coating layer 116 consists essentially of the tissue-growth-inhibiting polymer. In some implementations, the coating layer can comprise additional compounds.

In some implementations, coating layer 116 can comprise a woven or nonwoven fabric (distinct from fabric layer 112) of the tissue-growth-inhibiting polymer.

In some implementations, coating layer 116 can comprise a laminate (e.g., a film) of the tissue-growth-inhibiting polymer.

In some implementations, an outer surface of the coating layer (which can define first face 101a of sheet 100, first face 151a of sheet 150, and/or second face 151b of sheet 151b) can consist essentially of the tissue-growth-inhibiting polymer (e.g., can be a film of the polymer), while deeper within the coating layer a different material and/or structure can provide other characteristics, such as adhesion to binding layer 114, durability, and/or structural qualities.

In some implementations in which the tissue-growth-inhibiting polymer is a PTFE, coating layer 116 can comprise several layered sublayers (e.g., laminae) of the PTFE. In some implementations, these sublayers can be anisotropic, and can be arranged such that each of the sublayers is disposed orthogonally to an adjacent one of the sublayers, e.g., each sublayer alternating with the previous and the next sublayer. In some implementations, such a coating layer comprises fewer than ten such sublayers, e.g., at least two and/or no more than six sublayers (e.g., 2-6 sublayers, such as 2-5 sublayers), e.g., exactly 5 sublayers.

In some implementations, coating layer 116 is porous. In some implementations, coating layer is nonporous.

In some implementations, coating layer is 5-70 microns thick.

In some implementations, fabric layer 112 comprises a woven fabric. In some implementations, fabric layer 112 comprises a knitted fabric. In some implementations, fabric layer 112 comprises a braided fabric. In some implementations, fabric layer 112 comprises a nonwoven fabric. In some implementations, fabric layer 112 comprises a 3-D fabric. In some implementations, fabric layer 112 comprises an electrospun fabric. In some implementations, fabric layer 112 comprises a multiphasic fabric.

In some implementations, fabric layer 112 is 20-150 microns thick.

In some implementations, biding layer 114 comprises an acrylate. For example the binding layer can be essentially based on an acrylate compound such as the salts, esters, and conjugate bases of acrylic acid. In some implementations, the binding layer comprises a siloxane and/or a polysiloxane. In some implementations, the binding layer comprises a thermoplastic polyurethane (TPU).

In some implementations, the binding layer is 50-30 microns thick.

In methods 10a and 10b, a sandwich is formed in which each binding layer 114 is sandwiched between fabric layer 112 and a respective coating layer 116. In method 10a this sandwich is sandwich 102 (upper frame of FIG. 1A), and in method 10b this sandwich is sandwich 104 (upper frame of FIG. 1B). Subsequently, fabric layer 112 is bound to coating layer(s) 116 (e.g., by binding the binding layer to both the fabric layer and the coating layer(s)) by heating the sandwich and/or compressing the sandwich (lower frame of FIGS. 1A and 1B).

Methods 10c and 10d are similar to methods 10a and 10b, respectively, except that they each include two heat and/or pressure steps. Binding layer(s) 114 are disposed against fabric layer 112 (FIGS. 1c and 1d; frame A), and are then bound to the fabric layer by compressing and/or applying heat to the layers (frame B). Subsequently, coating layer(s) 116 are added, thereby sandwiching binding layer(s) 114 between fabric layer 112 and the coating layer(s) (frame C). At this point in method 10c, sandwich 102 can be considered to have been formed. At this point in method 10d, sandwich 104 can be considered to have been formed. Subsequently, the layered fabric layer 112, binding layer(s) 114 and coating layer(s) 116 (e.g., the sandwich) is heated and/or compressed to form the sheet (frame D).

For methods 10c and 10d, in the transition between frame A and frame B, plates can be pressed against the binding layer in order to apply the heat and/or pressure. For example, such plates can be, or can be similar to, any of plates 310, 320, 710, and 720 detailed hereinbelow. Such plates can be configured to not become bound to binding layer 114 despite binding layer being configured specifically to bind to other materials such as coating layer 116. The plates can be configured in this manner by having a highly smooth and/or non-porous surface. In contrast, coating layer 116 (or at least the inner surface thereof) can be sufficiently rough and/or porous to allow the binding layer to bind, e.g., by the heated and/or pressed binding layer entering microscopic pores and/or crevices. This can be the case even for implementations in which coating layer 116 has a similar chemical composition as the plates, e.g., both can comprise polytetrafluoroethylene (PTFE), e.g., with their different characteristics deriving from their structure.

In some implementations, methods 10a and 10b may be advantageous, e.g., due to the smaller number of steps compared to methods 10c and 10d.

In some implementations, methods 10c and 10d may be advantageous, e.g., for implementations in which heat transfer through the entire sandwich may otherwise be slow and/or uneven. For example, for implementations in which coating layer 116 is relatively thick and/or insulating, methods 10a and 10b may be less reliable than methods 10c and 10d at causing binding layer 114 to bind to fabric layer 112. Furthermore, for implementations in which when the sheet comprises a frame disposed between layers (e.g., as detailed hereinbelow), methods 10c and 10d can facilitate more accurate and/or stable positioning of the frame with respect to the other layers. For example, the position of the frame with respect to the fabric layer can first be fixed by at least part of the frame being sandwiched between the fabric layer and a binding layer that is bound to the fabric layer—and then a coating layer can be added to the other side of the binding layer.

In all of methods 10a-10d, when heat and pressure are used to bind the binding layer to another layer, the heat and pressure can be applied simultaneously, or at least overlap temporally. However, in some implementations pressure and heat can be applied separately (e.g., sequentially and/or alternatingly).

In some implementations, the compression during each of the heating and compression steps is at a pressure of 0.01-1 megapascals (MPa).

In some implementations, the heating during the heating and compression steps is to a temperature of 37-260 degrees Celsius (degrees c.).

In some implementations of methods 10a and 10b, the heating and compression step lasts for 100-500 seconds. In some implementations of methods 10c and 10d the first heating and compression step lasts 20-420 seconds, and/or the second heating and compression step lasts 100-500 seconds.

For example, the sheet can be formed by applying, once or more, (i) pressure of 0.01-1 MPa and (ii) temperature of 37-260 degrees C., e.g., for a duration of 20-420 seconds or 100-500 seconds.

In some implementations, pressure and heat can be applied to the sandwich simultaneously. That is, one or more intervals of applied pressure and heating can be applied.

In some implementations pressure and heat can be applied separately (e.g., sequentially and/or alternatingly). That is, separate pressure intervals and heating intervals can be applied. In some implementations both (i) separate heating intervals and compressing intervals, and (ii) combined heating and compressing intervals can be applied.

In some implementations, sheet 100 and/or sheet 150 (e.g., once coating layer 116, binding layer 114, and fabric layer 112 are bound to each other) can have structural characteristics that enable the sheet to function as an implant. For example, the sheet can be implanted (e.g., sutured and/or anchored to tissue) alone. Alternatively, and/or as described in more detail hereinbelow, an implant can be provided in which the sheet is attached to a frame.

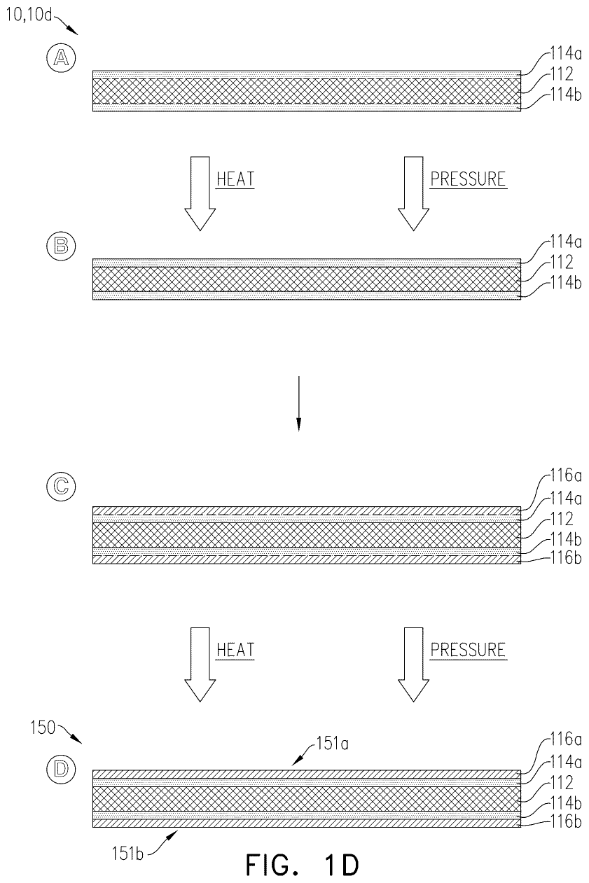

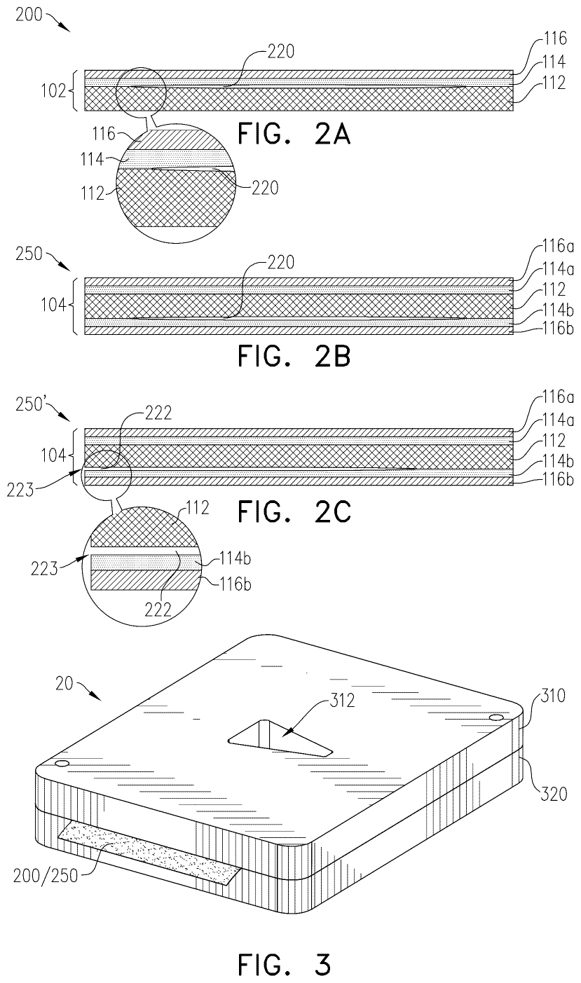

Reference is now made to FIGS. 2A, 2B and 2C which are schematic illustrations of sheets, such as sheet 200 and sheet 250, which can be considered to be variants of sheet 100 and sheet 150, respectively, in accordance with some implementations. Sheets 200 and 250 can be identical to sheets 100 and 150, respectively, except that each one of the sheets, sheet 200 and sheet 250, defines a pocket. In some implementations, a pocket can be defined between the fabric layer and the binding layer. For example, a pocket 220 can be disposed within sandwich 102. As illustrated in FIG. 2A, pocket 220 can be closed by the binding between fabric layer 112 and binding layer 114, which may circumscribe the pocket. FIG. 2B illustrates sheet 250, having five layers (e.g., pocket 220 can be disposed within sandwich 104), such that the pocket can be closed by the binding between fabric layer 112 and one of binding layers 114, such as second binding layer 114b, which may circumscribe the pocket.

In some implementations, the sheet can define an opening into the pocket, e.g., at least one edge of the pocket is open. FIG. 2C illustrates such an arrangement, in which a sheet 250′ (which can be considered to be a variant of sheet 250) has a pocket 222 disposed within sandwich 104, such that at an opening 223 fabric layer 112 is unbound from second binding layer 114b. Opening 223 can be disposed at an edge of sheet 250′, e.g., to enable insertion of one or more other component into the sheet. For example, in some implementations, a frame can be introduced into pocket 222 via opening 223, which may increase structural characteristics of the sheet. Similarly, one or more other components (such as a frame) can be included within pocket 220 by forming the sheet to include the frame, e.g., as described in more detail hereinbelow.

It should be noted that although the examples described with reference to FIGS. 2B and 2C refer to pocket disposed between fabric layer 112 and second binding layer 114b, the scope of the present disclosure includes alternatively or additionally defining a pocket between the fabric layer and first binding layer 114a.

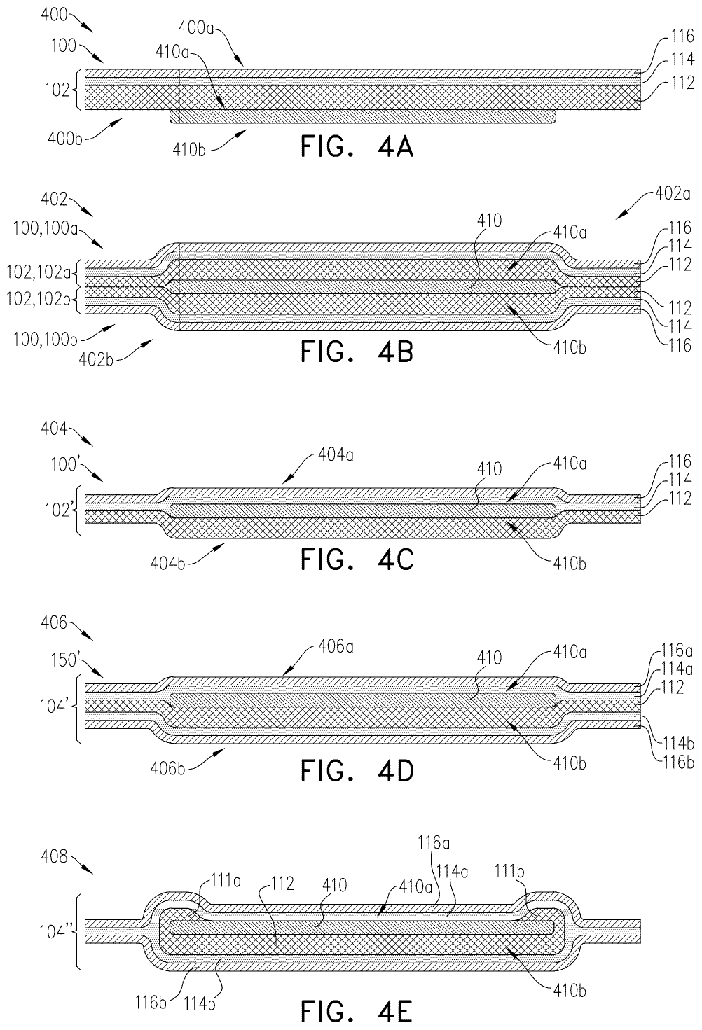

Reference is now made to FIG. 3, which is a schematic illustration of a laminating apparatus 20, in accordance with some implementations. The laminating apparatus comprises two plates, e.g., first plate 310 and second plate 320, configured to be pressed against each other such as to apply pressure and/or heat to a sandwich (e.g., sandwich 102 and/or 104), secured between the two plates, in order to form a sheet, e.g., sheet 200 and/or sheet 250. At least one of the two plates of laminating apparatus 20 can define a recess, such as recess 312 in a contact face of the plate. The recess, disposed at one of the plates can enable the formation of a pocket at sheet 200 and/or sheet 250.

As described hereinabove, sheets can be formed by compressing and/or heating sandwiched layers, which can cause the layers to bind to each other. The pocket can be formed by reducing (e.g., eliminating) this heating and/or compression at a discrete region, which will become a region of the pocket. For example, heat and/or compression can be applied to the sandwich around the region of the pocket, but not to the actual region of the pocket. Having a recess at one of the plates enables compressing and/or applying heat to other regions of the sheet (e.g., most of the sheet) while not compressing and/or applying heat to the region designated for the pocket. Accordingly, at the region of the designated pocket, the layers do not bind—and therefore a pocket is created at that region.

Recess 312 can be positioned and/or shaped according to the requirements of the shape and/or position of pocket 220 and/or 222. For example, in the illustrated example, recess 312 is disposed at a region (e.g., a central area) of plate 310, which, in some implementations, can correspond to the desired location of the pocket being at a central area of the sheet. Similarly, the shape of recess 312 is trapezoid which will accordingly result in a trapezoid-shaped pocket. When a different shape of a pocket is desired, recess 312 can be shaped accordingly. Additionally, when forming a pocket at an edge of the sheet such that the pocket can have an opening, e.g., opening 223, recess 312, can be moved from the center of the plate to an edge of the plate (or the sheet can be positioned differently between the plates).

Alternatively or additionally, formation of the pocket can be facilitated by an absence of a binding layer between the fabric layer and the coating layer. In some implementations, the binding layer can be pre-cut/pre-shaped to define a hole (which can be circumscribed by the rest of the binding layer and/or can be disposed at an edge of the binding layer). When the sandwich is formed, at the hole the binding layer is absent from between the fabric layer and the coating layer. Thus, when heat and/or pressure is applied in order to form the sheet, the fabric layer may not become bound to the coating layer at the hole in the binding layer.

In some implementations, a frame can be introduced into the pocket. For example, for implementations in which a pocket has an opening at an edge of the sheet, e.g., opening 223, a frame can be introduced via the opening into the pocket, subsequently to formation of the sheet (and the pocket). In some implementations, the pocket can be subsequently sealed, securing the frame therewithin—e.g., by closing opening 223, such as by again heating and/or applying pressure, and/or by other means such as stitching.

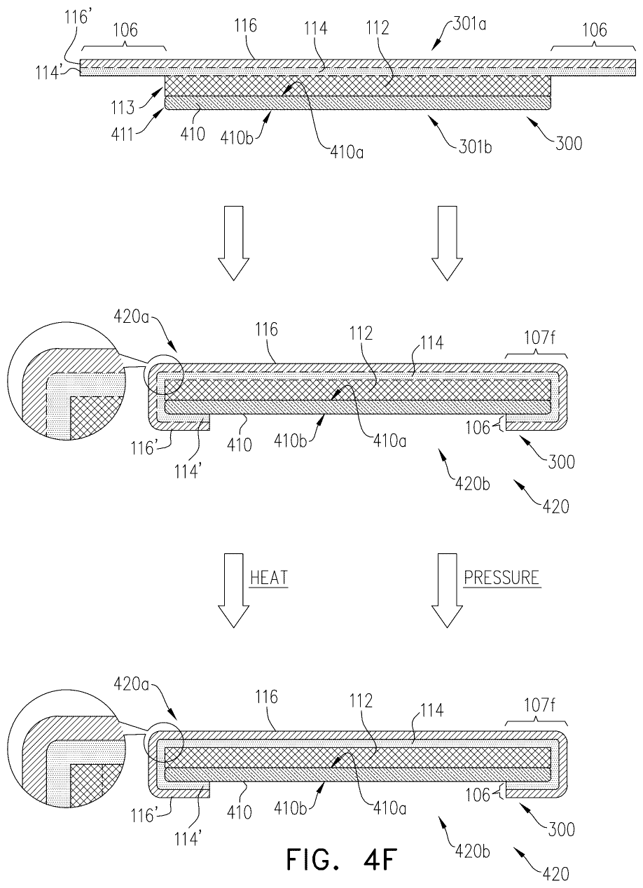

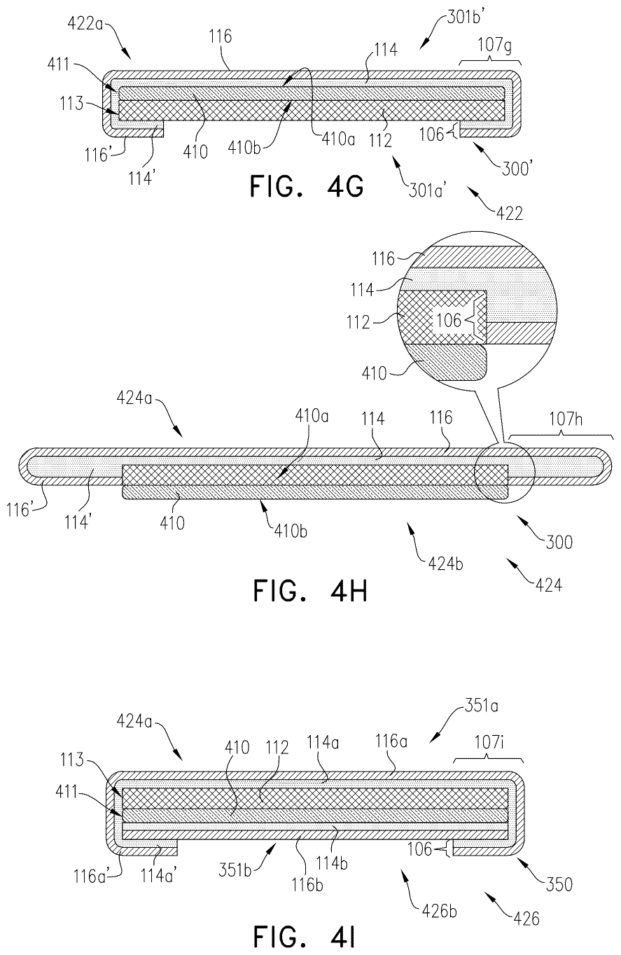

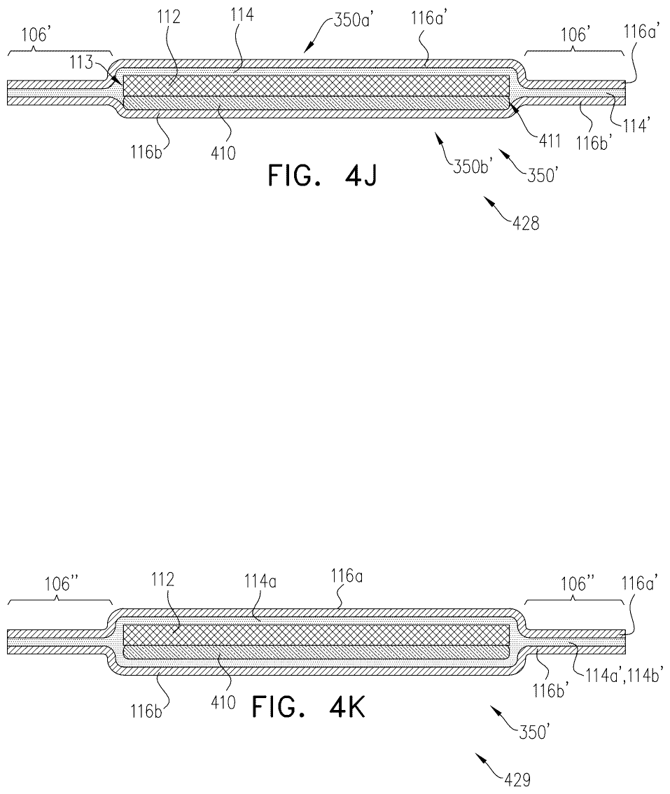

In some implementations in which a frame is disposed within the sheet, the frame can be positioned between two layers of the sandwich that will become the sheet (e.g., as illustrated hereinbelow with respect to FIGS. 4C to 4E), prior to application of pressure and/or heat to form the sheet. The pocket can therefore be formed with at least part of the frame already disposed in the region at which the pocket will be formed. For example, for implementations in which at a recess of at least one of the plates of laminating apparatus 20 defines a recess therein, the recess can be positioned over the frame so as to form the pocket at the frame. In some implementations, part of the frame extends beyond the boundaries of the pocket.

Reference is now made to FIGS. 4A to 4K, which are schematic illustrations of sheets 100, 150, 200, 250, 300 and 350 or variants thereof, being fixedly attached to a frame, in accordance with some implementations. In some implementations, a system, apparatus, implant, etc. can comprise a frame, e.g., a frame 410, such that the sheet can be fixedly attached to the frame in a manner that covers at least part of the frame. In some implementations, attaching a frame to a sheet, can increase the structural characteristics and/or durability of the apparatus, which can enable it to better function as an implant, e.g., a prosthetic valve, a stent and/or similarly to a leaflet-augmentation patch and/or as a wing as disclosed in one or more of the following references, each of which is incorporated herein by references in its entirety:
International Patent Application No. PCT/IB2021/060436 to Tennenbaum et al., filed Nov. 11, 2021, which published as WO 2022/101817;
International Patent Application No. PCT/US2021/039587 to Chau et al., filed Jun. 29, 2021, which published as WO 2022/006087;
International Patent Application No. PCT/US2022/052834 to Chau et al., filed Dec. 14, 2022.

In some implementations, an apparatus comprising a cardiovascular implant can be formed by fixedly attaching a sheet to a frame. The implant can be used as a prosthetic valve, a stent, a wing and/or a leaflet-augmentation patch. For example, the implant can be used at a valve of a heart of a subject, the valve having an annulus, a first leaflet and a second leaflet opposing the first leaflet, the heart having a chamber upstream of the valve. The implant can comprise, among other components, a frame and a sheet. The sheet can comprise, at least, a fabric layer secured against at least a portion of the frame, a coating layer, comprising a polymer configured to inhibit tissue growth thereon, and a binding layer, sandwiched between the fabric layer and the coating layer, and binding the fabric layer to the coating layer.

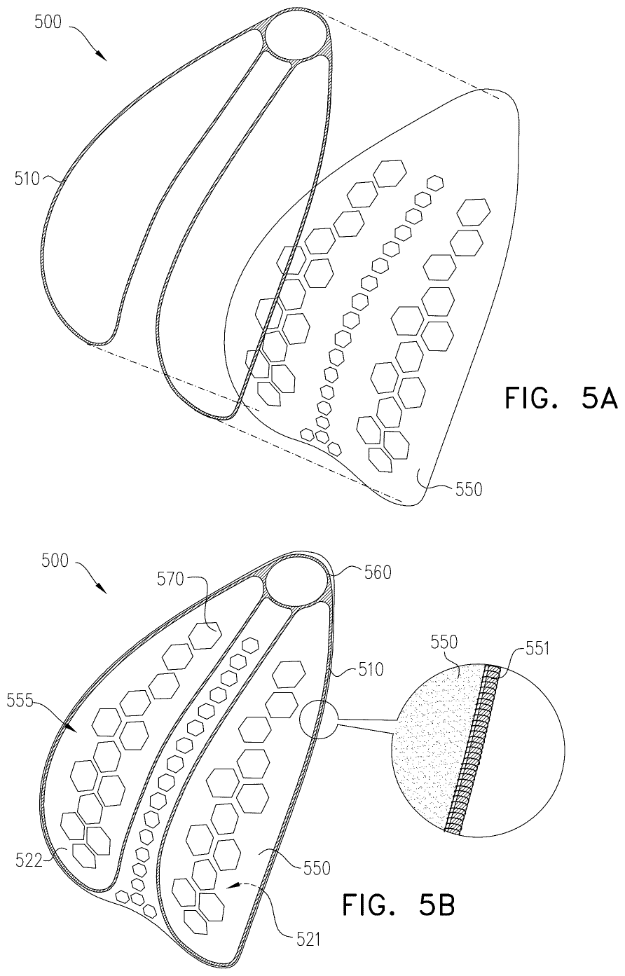

FIG. 4A schematically illustrates an implant 400 that comprises sheet 100 fixedly attached to frame 410, in accordance with some implementations. As indicated hereinabove with respect to FIGS. 1A-1B, first face 101a of the sheet, which may inhibit tissue growth thereon, can be placed adjacent to a tissue surface of a subject. Thus, first face 101a can form at least a portion of a first (e.g., contact) face 400a of implant 400. Respectively, for implant 400, frame 410 can be disposed adjacent to (e.g., against) fabric layer 112, e.g., at the second face of the sheet, forming at least a portion of a second (e.g., opposing) face 400b of implant 400, e.g., such that the frame is absent from contact face 400a of the implant. In some implementations, sheet 100 can be fixedly attached to the frame by stitching and/or gluing sheet 100 to frame 410, thereby forming implant 400, e.g., as detailed hereinbelow in FIGS. 5A and 5B with respect to implant 500.

It should be noted that frame 410 is intended to be generic, e.g., representing a frame of any implant, mutatis mutandis. For example, frame 410 may represent a frame used in implant 500 and/or implant 600 described herein, and/or a frame used in an implant described in one or more of the references incorporated herein by reference.

In some implementations, concealing the frame between one or more sheets and/or layers thereof may advantageously prevent the frame from encountering the surroundings of the implant, e.g., such as tissue, and can help protect the frame and/or inhibit tissue growth thereon, as well as protecting the tissue from injury. However, in some implementations, it can also be advantageous to avoid exposing a fabric layer of a sheet, e.g., in order to avoid any tissue growth on the implant. Therefore, in some implementations, in order to inhibit tissue growth on both faces of an implant, an additional coating layer, which can define the second face of the implant, can be included. FIGS. 4B, 4D, 4E4I, 4J and 4K schematically illustrate examples of such inclusion, in accordance with some implementations. In some implementations, an additional sheet 100 can be included to cover second side 410b of the frame, with coating layer 116 of the additional sheet defining the second face of the implant.

FIG. 4B schematically illustrates an implant 402 that comprises two sheets 100, e.g., a first sheet 100a and a second sheet 100b. Sheets 100a and 100b can be considered to be variants of sheet 100, e.g., identical to sheet 100 except where noted, connected to each other, in accordance with some implementations. Each one of sheets 100 can be formed independently, e.g., by heating and/or compressing each one of sandwiches 102a and 102b, and each one of the sheets can independently be fixedly attached, e.g., by being stitched and/or adhered, to frame 410. For example, each one of sheets 100 can be fixedly attached to a different side of the frame such that both sides of frame 410 can be covered by a fabric layer. Alternatively or additionally, the two sheets can be fixedly attached to each other, e.g., a first fabric layer 112 can be attached to a second fabric layer 112, for example by being stitched and/or adhered to each other, and/or to frame 410 such that the coating layers can define both faces of the implant, e.g., first coating layer 116 can define first face 402a and second coating layer 116 can define second face 402b. Alternatively or additionally, a single sheet 100 can be folded over an edge of frame 410 in order to cover both sides of the frame in a similar manner, mutatis mutandis.

Implant 402 can be formed by fixedly attaching sheets 100a and 100b to each other and/or to frame 410, positioned therebetween. For example, first sheet 100a (comprising first fabric layer 112, first binding layer 114 and first coating layer 116), can be fixedly attached, e.g., by being stitched and/or adhered, to frame 410 such that first coating layer 116 can define first face 402a and first fabric layer 112 can be adjacent to first side 410a of the frame. Second sheet 100b (comprising second fabric layer 112, second binding layer 114 and second coating layer 116), can be fixedly attached, e.g., by being stitched or adhered, to frame 410 such that second coating layer 116 can define first face 402a and second fabric layer 112 can be adjacent to second side 410b of the frame, thereby covering both sides of frame 410. In some implementations the first fabric layer of first sheet 100a can also be fixedly attached, e.g., by being stitched and/or adhered, to the second fabric layer of second sheet 100b, which may increase binding between the two sheets and the overall physical characteristics of implant 402. The combination of the two sheets, each being fixedly attached to frame 410 such that the two coating layers define both faces of the implant, e.g., first face 402 and second face 402b, may inhibit tissue growth at the faces of implant 402.

In some implementations, an additional binding layer can be added, e.g., adjacent to the frame, such that implant 402 can be formed by heating and/or compressing the additional binding layer between sandwiches 102a and 102b.

FIG. 4C schematically illustrates an implant 404 that comprises a sheet 100′ fixedly attached to frame 410, in accordance with some implementations. Sheet 100′ can be considered to be a variant of sheet 100, e.g., identical to sheet 100 except that sheet 100′ is formed from a sandwich 102′ (e.g., by heating and/or compressing sandwich 102′) rather than from sandwich 102. Sandwich 102′ can be considered to be a variant of sandwich 102, in which sandwich 102′ includes frame 410 sandwiched between other layers of the sandwich. For example, the other layers of the sheet (e.g., the layers common to both sheet 100 and sheet 100′) can be fixedly attached to the frame by the frame being sandwiched between fabric layer 112 and binding layer 114. Similarly to implant 400, detailed hereinabove with respect to FIG. 4A, a first face 404a of implant 404 can be defined by coating layer 116, which inhibits tissue growth thereon. Second face 404b of implant 404, opposite to first face 404a, can be defined by fabric layer 112, and therefore can enable tissue growth thereon. In some implementations, first face 404a can be positioned adjacent to a tissue surface of a subject.

In some implementations, the thickness of frame 410 between the fabric layer and the binding layer can be 20-500 microns.

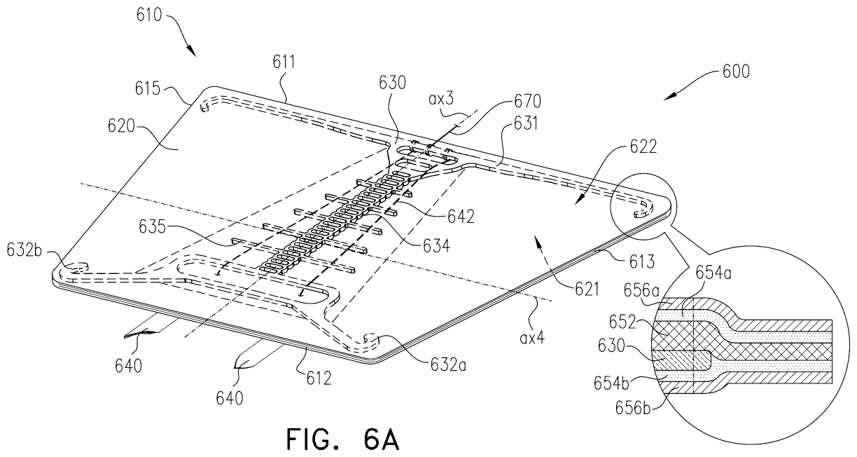

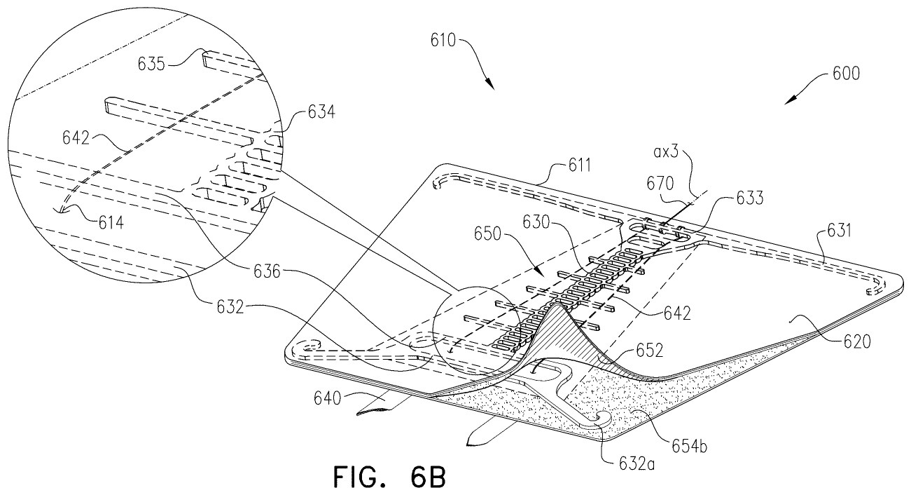

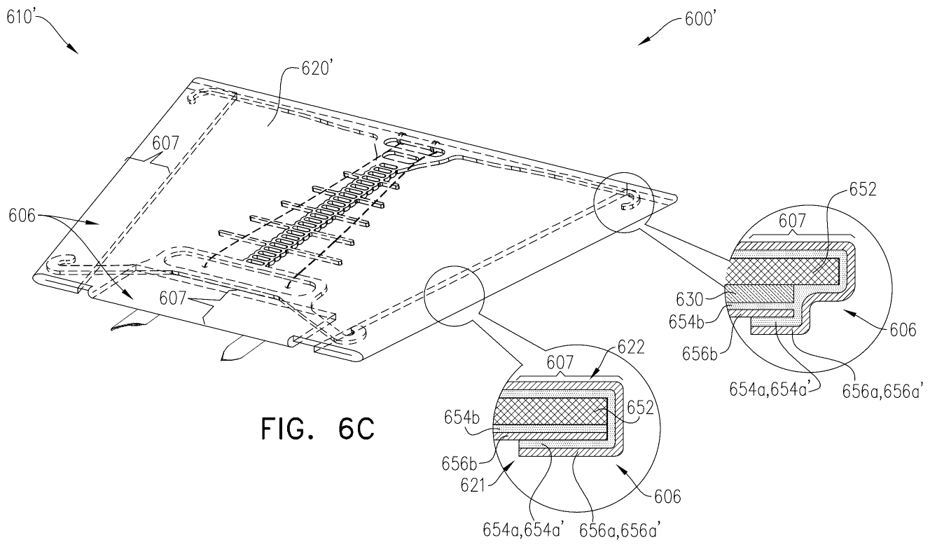

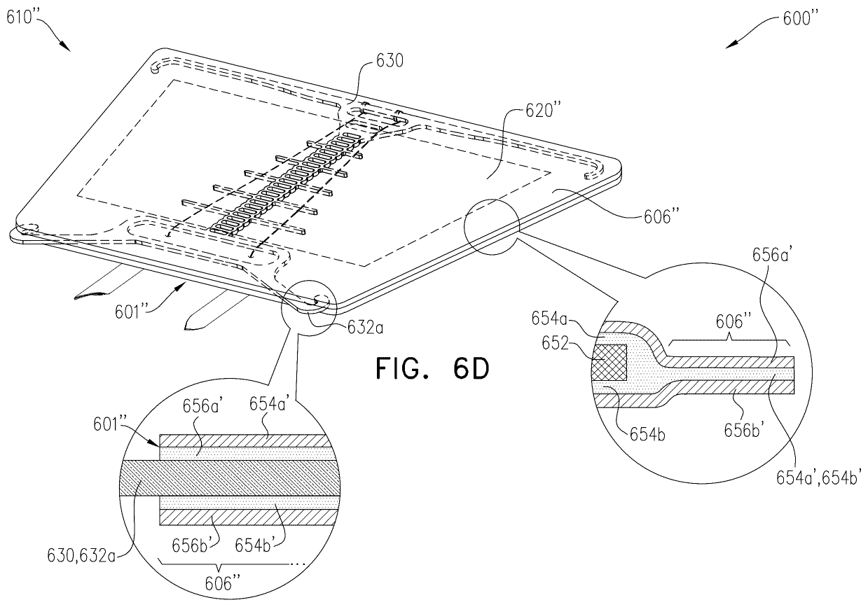

In some implementations, implant 404 can further comprise a pocket (e.g., as detailed hereinabove with respect to FIGS. 2A-C). For example, such a pocket can enable a cord to move (e.g., slide), within the pocket, with respect to frame 410. An example of such an application is described hereinbelow with reference to FIG. 6A.

FIG. 4D schematically illustrates an implant 406 that comprises a sheet 150′ fixedly attached to frame 410, in accordance with some implementations. Sheet 150′ can be considered to be a variant of sheet 150, e.g., identical to sheet 150 except that sheet 150′ is formed from a sandwich 104′ (e.g., by heating and/or compressing sandwich 104′) rather than from sandwich 104. Sandwich 104′ can be considered to be a variant of sandwich 104, in which sandwich 104′ includes frame 410 sandwiched between other layers of the sandwich. For example, the other layers of the sheet (e.g., the layers common to both sheet 150 and sheet 150′) can be fixedly attached to the frame by the frame being sandwiched between a binding layer (e.g., first binding layer 114a, as shown) and fabric layer 112. Thus, implant 406 can have a first face 406a and a second face 406b, both of which can be defined by a respective coating layer, e.g., first coating layer 116a and second coating layer 116b. The coating layers can be bound to fabric layer 112 by first binding layer 114a and by second binding layer 114b, sandwiched therebetween. Accordingly, implant 406 can have two faces that inhibit tissue growth thereon.

FIG. 4E, schematically illustrates an implant 408 that comprises a sheet 150″ fixedly attached to frame 410, in accordance with some implementations. Sheet 150″ can be considered to be a variant of sheet 150′, e.g., identical to sheet 150′ except that sheet 150″ is formed from a sandwich 104″, rather than from sandwich 104′. In sandwich 104″, and therefore in sheet 150″, a fabric layer 112′ is wrapped around frame 410 from the first side to the second side of the frame, such that at least part of the frame is sandwiched between parts of the fabric layer. For example, a first part 111a of the fabric layer can be disposed against second side 410b of the frame, and each place where the fabric layer is wrapped around the frame, a second part iiib can be disposed against first side 410a of the frame. In the example shown, there are two such places. That is, in the example shown, the fabric layer covers second face 410b of frame 410, and two parts iiib of the fabric layer are folded over the edges of first side 410a of the frame. In some implementations, this wrapping can contribute to the fixation of the frame to the fabric layer.

In some implementations, and as shown, a region 409 of first side 410a may not be sandwiched between parts of the fabric layer, and may therefore instead be sandwiched between the fabric layer and a binding layer (e.g., binding layer 114a).

In some implementations, the overall thickness of the implants described hereinabove, e.g., implants 400, 402, 404, 406 and/or 408, including the frame, can be 50-1000 microns, e.g., 200-500 microns.

In some implementations, for example, to reduce likelihood of an edge of the implant damaging and/or injuring a tissue of a subject, e.g., during intentional or unintentional movement of the implant along and/or against the tissue, the edges of the implant can be soft and/or can have an obtuse angle and/or a rounded shape.

FIGS. 4F, 4G, 4H, 4I, 4J and 4K schematically illustrate implants that can be considered to be variants of some of the implants detailed hereinabove. In some implementations, these implants can have round edges and/or soft edges. In some implementations, in order to provide a round edge, an implant can be arranged to have a cover, which can be disposed over/around the edge (e.g., a smooth continuous cover). In some implementations, to provide a soft edge, an implant can be arranged to have no fabric and/or no frame at the edge, e.g., at least a section of a periphery of the implant can be absent of the fabric layer and/or any frame. For example, the implant can have a lip extending beyond the edge of the fabric layer. In some implementations the lip is folded to provide a rounded edge. In some implementations the lip is more flexible and/or softer than regions of the implant in which the fabric is disposed, and in some implementations the lip is not folded.

In some implementations, the coating layer and the binding layer can extend together beyond an edge of the fabric layer to define a lip in which a lip-portion of the coating layer and a lip-portion of the binding layer are disposed. For example, for implementations in which the coating layer defines a first face of the sheet (such as first face 101a and first face 151a of sheets 100 and 150, respectively), and in which the sheet has a second face opposite the first face, the lip can be folded onto the second face, e.g., such that the lip-portion of the binding layer is sandwiched between the lip-portion of the coating layer and the second face.

In some implementations, the lip can be folded to define a hem of the sheet. In some implementations, the hem can be formed by folding the lip around the edge of the fabric layer, such that within the hem the fabric layer is be disposed between the lip-portion of the binding layer and another portion of the binding layer (optionally along with part of the frame also disposed between the lip-portion of the binding layer and the other portion of the binding layer).

FIG. 4F schematically illustrates an implant 420 that comprises a sheet 300 fixedly attached to frame 410 such that the implant has two rounded edges, in accordance with some implementations. Sheet 300 can be considered to be a variant of sheet 100′, e.g., identical to sheet 100′ except that in sheet 300 coating layer 116 and the binding layer 114 extend together beyond an edge 113 of fabric layer 112, thereby defining a lip 106 in which a lip-portion 116′ of the coating layer and a lip-portion 114′ of the binding layer are disposed. In some implementations, and as shown, lip 106 can extend beyond an edge 411 of frame 410.

The upper image of FIG. 4F shows sheet 300 prior to formation of the rounded edges, the middle image shows lips 106 have been folded to form hems 107f, which provide the rounded edges, and the lower image shows the sheet having been fixed in this arrangement by heat and/or pressure treatment, as described hereinabove, mutatis mutandis.

Coating layer 116 can define a first face 301a of the sheet, the sheet having a second face 301b opposite to the first face (upper image). As illustrated, lip 106 can be folded onto second face 301b, such that lip-portion 114′ of binding layer 114 becomes disposed between (i) lip-portion 116′ of the coating layer and (ii) second face 301b of the sheet (middle image). For example, and as shown, lip portion 114′ of the binding layer can be sandwiched between (i) lip-portion 116′ of the coating layer and (ii) frame 410. Additionally or alternatively, at least part of frame 410 can be disposed between (i) lip-portion 114′ of the binding layer and (ii) the fabric layer 112, e.g., as shown. As illustrated, implant 420 comprises a first lip and a second lip, such that each one of lips 106 can be folded around a corresponding edge 411 of the frame, thereby defining a corresponding hem(s) 107f. In some implementations, the edge of the frame can be disposed within hem(s) 107f, e.g., between the lip-portion(s) of the binding layer and another portion of the binding layer. In some implementations, at least a part of the fabric layer can further be disposed within the hem(s).

In some implementations, the frame can be attached to the second side of the sheet, e.g., by the lip(s). For example, the lips(s) can be folded over the edge(s) of the frame and fixed to the frame and/or to the second side of the sheet, e.g., by heating and/or pressing the lip(s) against the frame and/or to the second side of the sheet, e.g., hem(s) 107f, as described hereinabove mutatis mutandis. In some implementations, at least part of the frame can be fixedly attached to the sheet by adhesive and/or by stitches. Additionally or alternatively, stiches may pass from the lip through the frame to the second side of the sheet, or vice versa, thereby stitching the frame to the sheet and the lip simultaneously.

In some implementations, having coating layer 116 define first face 420a of the implant can inhibit tissue growth thereon. Furthermore, folding of lips 106 over edges of the frame and the fabric layer, can shape the implant so as to have smooth edges. Additionally, by such folding over onto second face 310b of the sheet, the lip-portions of the coating layer can provide tissue-growth inhibition also to part (or even all) of second face 420b of the implant.

In some implementations, although not illustrated, an implant can be provided with rounded edges by forming a hem by folding also fabric layer 112 (e.g., wrapping the fabric layer around the frame). Such an implant can, for example, resemble implant 400 (FIG. 4A), but with the parts of the sheet (e.g., fabric layer 112, binding layer 114, and coating layer 116) that extend beyond the frame being wrapped around the edges of the frame. In some implementations, the sheet can be secured by stitches and/or adhesive. In some implementations, the sheet can cover first side 410a of frame 410 and part of the fabric layer can be disposed against second side 410b of the frame, thereby creating a hem. In some implementations, multiple such hems can be formed (e.g., contributing to the fixing of the frame to the sheet) e.g., while leaving at least a majority of second side 410b of the frame exposed.

FIG. 4G schematically illustrates an implant 422 that comprises a sheet 300′ fixedly attached to frame 410 such that the implant has two rounded edges, in accordance with some implementations. Sheet 300′ can be considered to be a variant of sheet 300, e.g., identical to sheet 300 except that in sheet 300′ frame 410 is sandwiched between fabric layer 112 and binding layer 114. In sheet 300′ coating layer 116 and the binding layer 114 extend together beyond an edge 113 of fabric layer 112, thereby defining a lip 106 in which a lip-portion 116′ of the coating layer and a lip-portion 114′ of the binding layer are disposed. In some implementations, and as shown, lip 106 can extend beyond an edge 411 of frame 410.

Coating layer 116 can define a first face 301a′ of the sheet, the sheet having a second face 301b′ opposite to the first face. As illustrated, lip 106 can be folded onto the second face, such that lip-portion 114′ of binding layer 114 can be disposed between (i) lip-portion 116′ of the coating layer 116 and (ii) second face 301b of the sheet. In the illustrated example, lip portion 114′ of the binding layer is sandwiched between (i) lip-portion 116′ of the coating layer and (ii) fabric layer 112. Additionally or alternatively, at least part of fabric layer 112 can be disposed between (i) lip-portion 114′ of the binding layer and (ii) the frame 410, e.g., as shown. As illustrated, implant 420 comprises a first lip and a second lip, such that each one of lips 106 can be folded around a corresponding edge 113 of the fabric layer, thereby defining a corresponding hem(s) 107g. In some implementations, the edge of the fabric layer can be disposed, within the hem(s) of the implant, e.g., between the lip-portion(s) of the binding layer and another portion of the binding layer. In some implementations, at least a part of the fabric layer can frame be disposed within the hem(s).

In some implementations, the folded lips can become attached to fabric layer 112 at the second side of the sheet. For example, the lips(s) can be folded over the edge(s) of the fabric layer and fixed to the fabric layer (e.g., the second side of the sheet), e.g., by heating and/or pressing the lip(s) against the fabric layer and/or to the second side of the sheet, e.g., hem(s) 107g, as described hereinabove mutatis mutandis. In some implementations, at least part of hem(s) 107g can be fixedly attached to the sheet by adhesive and/or by stitches. Additionally or alternatively, stiches can pass from the lip through the fabric layer and/or the frame, or vice versa, thereby stitching the fabric layer and/or frame to lip(s).

FIG. 4H schematically illustrates an implant 424 that comprises a sheet 300 fixedly attached to frame 410 such that the implant has two rounded edges, in accordance with some implementations. Implant 424 can be considered to be a variant of implant 420, e.g., identical to implant 420 except that implant 424 has hems 107h that are formed by having lip(s) 106 being folded onto themselves (rather than over the edge(s) of the frame and/or the fabric layer). For example, in hems 107h, lip-portions 114′ of the binding layer can be sandwiched between lip-portions 116′ of the coating layer. Hems 107h can be fixed by applying heat and/or pressing on to the hems, e.g., as described hereinabove, mutatis mutandis.

In some implementations, part of second face 424b of implant 424 can be provided (e.g., defined) by hems 107h. In some implementations, hems 107h can be arranged such that lip-portion(s) 116′ of the coating layer extend back to fabric layer 112, e.g., such that an edge of each lip-portion 116′ faces and/or is adjacent to a respective edge 113 of the fabric layer.

It should be noted that a variant of implant 424 can be formed by reversing the positions of frame 410 and fabric layer 112, such that the frame is sandwiched between the binding layer and the fabric layer.

FIG. 4I schematically illustrates implant 426 that comprises a sheet 350 fixedly attached to frame 410 such that the implant has two rounded edges, in accordance with some implementations. Sheet 350 can be considered to be a variant of sheet 150′, e.g., identical to sheet 150′ except that in sheet 350 the first coating layer and the first binding layer extend together beyond edge(s) 113 of the fabric layer, thereby defining lip(s) 106 in which lip-portion(s) 116a′ of the first coating layer and lip-portion(s) 114a′ of the first binding layer are disposed. In some implementations, at at least part of the sheet, the lip(s) can also extend beyond edge(s) 411 of the frame, e.g., as shown. First coating layer 116a can define a first face 351a of the sheet, and at least a portion of a second face 351b of the sheet, opposite to the first face, can be defined by second coating layer 116b. Lips 106 are folded onto second coating layer 116b, such that the lip-portions 114a′ of the first binding layer are sandwiched between lip-portions 116a′ and the second coating layer. Accordingly, the folding of lips 106 defines hems 107i of the sheet. Thus, in some implementations, second face 426b of the implant can be defined partly by second coating layer 116b and partly by lip-portions 116a′ of the first coating layer. In some implementations, the entirety of second face 426b can therefore inhibit tissue growth thereon.

In some implementations, at least a part of the frame and/or of the fabric layer can be disposed within hems 107i. In some implementations, the second face of the frame can be covered by the second binding layer and the second coating layer.

It should be noted that folding the lips to create hems of the implant may provide additional benefits (additional to having rounded edges) to the implant. For example, hems provide a continuous surface of the coating layer from the first face to the second face (e.g., on which they are folded on) of the implant thereby concealing edges of the fabric layer and/or edges of the frame. Having the fabric layer and/or the frame fully disposed within the sheet, e.g., along with the edges of the fabric layer and/or of the frame, may inhibit tissue growth along the edges of the fabric layer and/or of the frame. Concealment of the edges of the layers by forming hems may additionally or alternatively inhibit separation of the layers at their edges (e.g., at the edge of the sheet), e.g., delamination, fraying, peeling, or flaking that may result from intermittent and/or recurring contact with tissue over time.

A similar advantageous feature (e.g., inhibiting tissue growth by concealing edges of layers comprising the sheet) can also be achieved by having two coating layers and at least one binding layer extending beyond the edges of the fabric and/or frame thereby defining lips of the sheet/implant (e.g., without being folded to form hems) as detailed hereinbelow with respect to FIGS. 4J and 4K.

In some implementations, lips 106 may not be folded onto second coating layer 116b. For example, second coating layer 116b may not extend sufficiently close to the edges of frame 410 and/or the edges of fabric layer 112 for the lips to reach the second coating layer. Some such arrangements can resemble implant 420 and/or implant 422 with the addition of a second coating layer and a second binding layer at the second face of the implant, between lips 106.

It should be noted that for any one of implants 4F to 4I, the sheets can be formed, e.g., by heating and/or compressing the relative sandwich, before folding the lip(s) onto the second face of the sheet. In some implementations, the lip(s) can first be folded onto the second side of the sandwich before heat and/or compression pressure is applied, which upon heating and/or compressing the sandwich along with the hems can form a sheet having rounded edges.

FIG. 4J schematically illustrates an implant 428 that comprises a sheet 350′ fixedly attached to frame 410 such that the implant has lips 106′, in accordance with some implementations. Sheet 350′ can be considered to be a variant of sheet 150′ (e.g., identical to sheet 150′) except that in sheet 350′ the first coating layer, the second coating layer, and the first binding layer extend together beyond one or more edges of the fabric layer and/or of the frame, thereby defining lips 106′ in which lip-portions 116a′ of the first coating layer, lip-portions 116b′ of the second coating layer and lip-portions 114′ of the binding layer are disposed. Lip(s) 106′ can be softer and/or more flexible than the other portions of sheet 350′, e.g., portions in which fabric layer 112 is disposed. It is to be noted that, whereas in implants 420, 422 and 426 lips 106 are folded, lips 106′ are not folded, and serve as lips of the final implant, e.g., they define edges of the implant.

In some implementations, within lip 106′, the lip-portion 116a′ of the first coating layer can be bound to lip-portion 116b′ of the second coating layer by lip-portion 114′ of the first binding layer. For example, the lip(s) can be heated and/or compressed, along the rest of the sandwiched layers, e.g., upon formation of sheet 350′ and/or implant 428. Having the fabric layer and/or the frame fully disposed within the sheet, may inhibit tissue growth thereon, e.g., even along the edges of the fabric layer and/or of the frame.

FIG. 4K shows a sheet 350″ of an implant 429, in accordance with some implementations. Sheet 350″ can be identical to sheet 350′ except for the inclusion of a second binding layer 114b. That is, sheet 350″ comprises a first binding layer 114a (which corresponds to binding layer 114′ of sheet 350′), and a second binding layer 114b. A lip-portion 114b′ of second binding layer 114b can extend, e.g., together with lip-portion 116b′ of the second coating layer, beyond the edge(s) of fabric layer 112 and/or the edge(s) of frame 410 into the lip(s). For example, within each lip 106″, lip-portion 116a′ of the first coating layer can be bound to lip-portion 116b′ of the second coating layer by both lip-portion 114a′ and 114b′ (of the first binding layer and the second binding layer). The binding of the lip-portions of the first coating layer to the lip-portions of the second coating layer by the lip-portions of the first and second binding layers, can occur as part of the overall formation of sheet 350″, e.g., by heating and/or compressing lips 106″ along with the rest of the sandwiched layers.

In some implementations, for any of the implants described hereinabove as including a frame, the frame can extend/protrude beyond an edge of the sheet. For example, the edge of the sheet beyond which the frame can protrude can be defined by any one of the layers, e.g., the first and/or second coating layer(s), the first and/or second binding layer(s) and/or the fabric layer. An example of this is shown in FIG. 6D.

In some implementations, the shape of the frame, supporting and/or attached to the sheet, can determine the shape of the implant.

In some implementations, although not illustrated, any one of the implants described hereinabove can be formed and/or function without a frame. Purely as an example, implants comprising a sheet, but no frame, may nonetheless include one or more folded lips (e.g., as described with reference to FIGS. 4F-I) and/or one or more non-folded lips (e.g., as described with reference to FIGS. 4J-K), mutatis mutandis. In some implementations, the shape of the implant may be determined by the shape the sheet is formed, cut and/or folded.

Reference is now made to FIGS. 5A and 5B, which are schematic illustrations of an implant 500 for use with a valve of a heart of a subject, in accordance with some implementations. FIG. 5A shows an exploded illustration of implant 500, while FIG. 5B illustrates the implant as a whole, in accordance with some implementations. Implant 500 can be anchored to a tissue of the heart so as to repair the function of the native leaflet. Implant 500 can be considered to be a variant of and/or can be similar, at least in its general purpose, to implant(s) disclosed in International Patent Application PCT/US2021/039587 to Chau et al., filed Jun. 29, 2021, which published as WO 2022/006087; and/or International Patent Application PCT/US2022/052834 to Chau et al., filed Dec. 14, 2022, each of which is incorporated herein by reference, mutatis mutandis. Similarly, the techniques described herein can be used, mutatis mutandis, to form any of the implants described in these references.

In some implementations, the implant can be shaped as a wing defining a contact face 521, and an opposing face 522 opposite to the contact face. The wing can be configured to be anchored to a tissue of a heart of a subject having a first leaflet and a second opposing leaflet, such that the wing can extend over the first leaflet toward the second leaflet, and the contact face faces the first leaflet.

In some implementations, implant 500 can comprise, among other component, a frame 510 and a sheet 550. Sheet 550 can be, or can comprise, any of the sheets described herein, such as sheets 100, 150, 200, 250, 300, 350, and/or their variants (e.g., comprising at least one fabric layer, at least one coating layer, and at least one binding layer). Frame 510 can be considered to be a variant of frame 410, mutatis mutandis.

Sheet 550 can be fixedly attached to the frame thereby defining a wing 555. In the illustrated example, sheet 550 is stitched to frame 510 by stiches 551, e.g., the sheet was formed and subsequently stitched to the frame. Edges of sheet 550 can be folded over edges of the frame to facilitate this stitching. It is to be noted, however, that sheet 550 can alternatively or additionally be attached to frame 510 by other means described herein, including the use of adhesive and/or the use of hems such as those described elsewhere herein.

The wing of implant 500 can be attached to an anchor receiver 560, configured to receive an anchor that is used to anchor the implant to tissue, such as to an annulus of a valve of a heart of a subject. The wing can define a contact face, which can be defined by a coating layer, e.g., contact layer 116/116a or contact layer 116b, and an opposing face opposite to the contact face.

Frame 510 can be flexible, such that the wing can have elastic and/or shape memory characteristics (e.g., the frame can be formed from and/or comprise nitinol, cobalt chrome, stainless steel, and/or a polymer), such that it can be folded or rolled so as to be delivered into a chamber of the heart in a shaft which can pass through a catheter, and can automatically expand to its resting position, e.g., resting and/or original shape, once deployed into the chamber of the heart.

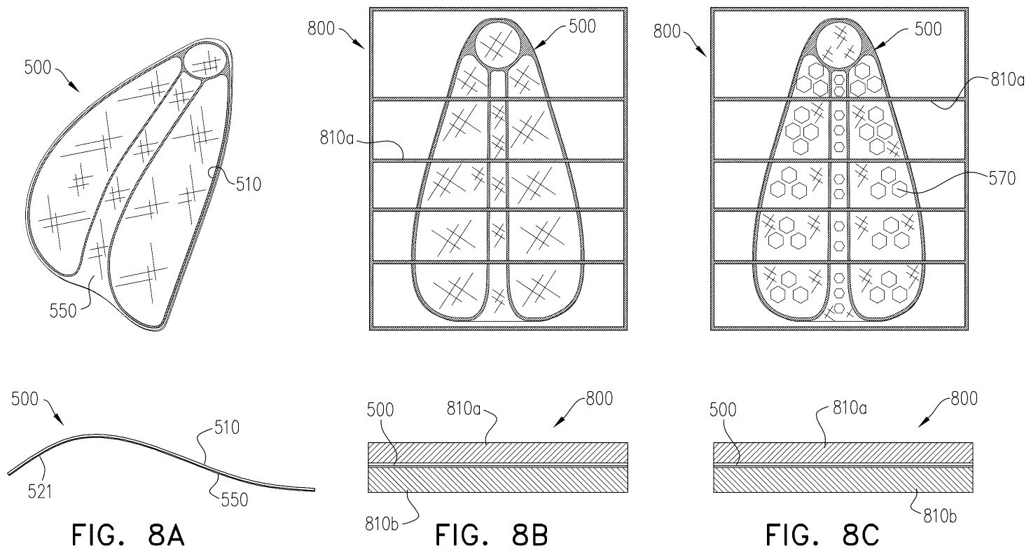

Wing 555 (e.g., sheet 550 thereof) can have multiple holes 570 therethrough, e.g., so as to facilitate blood flow through the wing. For example, after forming the wing from the sheet and frame, holes 570 can be formed, e.g., cut, through the implant and/or the wing, e.g., by hand, mechanically or by laser, as disclosed hereinbelow with respect to FIGS. 8A to 8C. In some implementations, holes 570 can be cut prior to attachment of sheet 550 to frame 510.

In some implementations, an implant can be shaped to define a leaflet-augmentation patch, configured to be attached to a leaflet of a valve of a heart of a subject. For example, after forming sheet 100 and/or sheet 150, the implant and/or wing can be cut, e.g., by hand, mechanically or by laser, so as to be shaped similarly to a leaflet-augmentation patch, e.g., as disclosed in International Patent Application PCT/IB2021/060436 to Tennenbaum et al., filed Nov. 11, 2021, which published as WO 2022/101817, and which is incorporated herein by reference. In some implementations, the shape of the frame, supporting and/or attached to the sheet, may determine the shape of the implant.

Reference is now made to FIGS. 6A to 6D which are schematic illustrations of an implant 600 and variants thereof, in accordance with some implementations. Implant 600 comprises a leaflet-augmentation patch 610, and can further comprise one or more patch anchors 640, configured to anchor the patch to a leaflet of a valve of a heart of a subject. Patch 610 can comprise a flexible sheet 620, fixedly attached to a frame 630. Sheet 620 can be, or can comprise, any of the sheets described herein, such as sheets 100, 150, 200, 250, 300, 350, and/or their variants (e.g., comprising at least one fabric layer, at least one coating layer, and at least one binding layer). Frame 630 can be considered to be a variant of frame 410, mutatis mutandis.

In some implementations, implant 600 and/or the variants thereof described herein can correspond to and/or can be variants of one or more upstream/downstream assemblies described in International Patent Application (PCT) Publication WO 2022/101817 to Tennenbaum et al., such as “upstream assembly 600”, “downstream assembly 700” and/or variants thereof. For example, implant 600 can be used in place of “upstream assembly 600” of Tennenbaum et al. (and/or variants described in Tennenbaum et al.), and/or patch 610 can be used in place of “patch 610” of Tennenbaum et al. (and/or variants described in Tennenbaum et al.). Similarly, the techniques described herein can be used, mutatis mutandis, to form any of the upstream assemblies and/or patches described in Tennenbaum et al.

In the illustrated example, and as described in more detail hereinbelow, sheet 620 is fixedly attached to frame 630 by the frame being sandwiched between (other) layers of the sheet, e.g., between a fabric layer 652 of the sheet and a binding layer of the sheet (e.g., a second binding layer 654b of the sheet). Thus, in some implementations, frame 630 can be considered to be a component of sheet 620.

Frame 630 can be flexible, such that patch 610 can be responsive to conditions in the heart, e.g., to the shape of one or both leaflets, in order to facilitate optimal coaptation. In some implementations, frame 630 (and thereby patch 610) can be configured to be more flexible on one axis than on another axis. For example, frame 630 can provide greater flexibility along a root-to-tip axis ax3 of patch 610 (e.g., with a tip 611 of the patch moving with respect to a root 612 of the patch), than along a mediolateral axis ax4 of the patch, transverse to the root-to-tip axis (e.g., with one lateral edge 613 moving with respect to the other lateral edge 615). Frame 630 can comprise a root brace 632 having a first root portion 632a and a second root portion 632b, which can extend at root 612, e.g., first root portion 632a at lateral edge 613 and second root portion at the other lateral edge 615. Frame 630 can define a tip brace 631, which can comprise a beam that extends substantially along tip 611, e.g., from one lateral edge 613 to the other lateral edge 615. Tip brace 631 and root brace 632 can provide patch 610 with a degree of mediolateral rigidity.

Frame 630 can comprise a spring 634, which can run between tip brace 631 and root brace 632 comprising lateral protrusions 635. In the example shown, spring 634 runs substantially along a root-to-tip midline of patch 610 (e.g., along axis ax3, if axis ax3 is a central root-to-tip axis). However, other positions and shapes are also possible.

Implant 600 can comprise one or more patch anchors 640 configured to anchor patch 610 (and thereby implant 600) to tissue of a heart of a subject, such as to a leaflet of a valve of the heart. In some implementations, one or more cords 642 couple patch anchors 640 to patch 610. For example, and as shown, each patch anchor can be coupled to the patch by a respective cord. Rather than each cord 642 being secured to patch 610 (e.g., frame 630 thereof) close to a point 614 at which the cord reaches the patch, the cord passes into the patch at a point 614, and extends along the patch to a site (e.g., a site on frame 630) to which it is secured. In the example shown, each cord 642 extends (e.g., in a root-to-tip direction, such as substantially parallel with axis ax3) to tip brace 631, to which it is secured.

In some implementations, each cord 642 extends along the patch within sheet 620 (e.g., between layers of the sheet), e.g., as shown. Having cord 642 extend within sheet 620 (e.g., rather than along the outside of the sheet) advantageously provides the sheet, and thereby patch 610, with smooth surfaces for optimal contact with tissue.

In the example shown, sheet 620 comprises (i) a first coating layer 656a bound to fabric layer 652 by a first binding layer 654a that is sandwiched between the first coating layer and the fabric layer, and (ii) a second coating layer 656b, on an opposite side of fabric layer 652 (and of frame 630) from coating layer 656a, which is bound to the fabric layer by a second binding layer 654b that is sandwiched between the second coating layer and the fabric layer. Second coating layer 656b defines a second face of sheet 620—and, accordingly, contact face 621 of implant 600 (e.g., a face that is placed in chronic contact with tissue). First coating layer 656a defines a first face of sheet—and, accordingly, an opposing face 622 of implant 600 (e.g., a face that can be placed in intermittent and/or recurring contact with tissue). At parts of patch 610′ in which frame 630 is disposed, the frame can be sandwiched between second binding layer 654b and fabric layer 652, e.g., as shown.

Spring 634 provides patch-anchor tightening functionality to implant 600 and variants thereof. Implant 600 is anchored to tissue by driving patch anchors 640 into (and typically through) the tissue. For example, implant 600 can be anchored to a heart valve leaflet that patch 610 is to augment, by driving patch anchors 640 through the leaflet to a second side of the leaflet, while the patch remains on the first side of the leaflet. Therefore, as patch anchors 640 are driven through to the second side of the leaflet (e.g., by drivers of a delivery tool for implant 600), the patch anchors are pushed away from patch 610, which remains at the first side of the leaflet. This tensions cords 642, which places spring 634 under stress (e.g., by pulling tip brace 631 toward root brace 632). The resulting straining (e.g., compression) of spring 634 allows cords 642 to be pulled out of patch 610 (e.g., through points 614), thereby allowing patch anchors 640 to be driven away from the patch and through the leaflet. In some implementations, and as shown, this can be achieved by a temporary reduction of a root-to-tip length of the patch—e.g., linearly contracting the patch.

Upon the release of patch anchors 640 (e.g., from the drivers) on the second side of the leaflet, spring 634 returns toward its original (e.g., resting) state, pulling the patch anchors back toward patch 610, and thereby securing the patch to the leaflet by sandwiching the leaflet between the patch anchors and the patch. It is hypothesized that providing the upstream assembly with self-tightening patch anchors such as these (e.g., by including the patch with a spring that is biased to draws the patch anchors tight) can advantageously obviate a need for a discrete tightening or locking step to be performed, and/or for a discrete locking component to be included.

Because cords 642 are disposed between layers of sheet 620, this self-tightening functionality may require that the cords are slidable between these layers, e.g., to be slid in one direction by the driving of the anchors, and to be slid in an opposite direction by the return of the spring toward its original (e.g., resting) state.

In some implementations, and as shown, spring 634 is coupled to root brace 632 via a patch-anchor support 636, defined by frame 630 and can be positioned and shaped to partially or completely surround points 614. Patch-anchor support 636 can advantageously provide one or more of the following benefits: (1) to serve to reinforce the patch at point 614, e.g., to protect against cord 642 cutting through sheet 620; (2) to serve as a bearing surface over which cord 642 slides as it moves through point 614; and/or (3) to provide opposition (e.g., an opposing force) against which patch anchors 640 can press the leaflet, thereby improving sandwiching of the leaflet between the patch anchors and patch 610, e.g., compared to a similar patch in which the patch anchors press the leaflet against part of the patch in which sheet 620 is not supported by frame 630.

In some implementations, a tether 670 that tethers the upstream assembly to the downstream assembly can be attached to a tip region of the patch. For implementations in which the patch comprises a frame that defines a tip brace, the tether can be attached to the tip brace. For example, for patch 610 tether 670 can be attached to tip brace 631 (e.g., to an eyelet defined by the tip brace), at the middle of tip 611. It is to be noted that, although tether 670 and cords 642 are both attached to tip brace 631, they act at least partly independently, e.g., with spring 634 tensioning cords 642 irrespective of tension on the tip brace. Furthermore, tip brace 631 can be configured to distribute at least some of the force exerted on patch 610 by tether 670 (e.g., during ventricular systole) across the width of the patch. Nonetheless, the finite length of cords 642 can limit the degree to which such force is experienced by patch 610 itself (e.g., by sheet 620), e.g., preventing the patch from being stretched deleteriously. That is, once cord 642 pulls its patch anchor tight against the leaflet, firmly sandwiching the leaflet against contact face 621 of patch 610, the cord inhibits further distancing of tip brace 631 from root brace 632.

As described hereinabove, the self-tightening functionality of patch 600 may require that cords 634 are slidable between the layers of sheet 620. Patch 600 can therefore be provided with features, and/or manufactured using techniques that facilitate smooth sliding of the cords between the layers of the sheet.

In some implementations, such smooth sliding is facilitated by frame 630 being shaped, dimensioned, and/or positioned to separate (e.g., to limit or prevent contact between) cords 642 and one or both binding layers 654, which may be inherently tacky and may otherwise grip the cords. For example, frame 630 can define lateral protrusions 635, which can extend laterally from spring 634 to protrude between cords 642 and a binding layer. In the example shown, protrusions 635 extend between and separate cords 642 and second binding layer 654b. In the example shown, similar protrusions are not provided on the other side of cords 642 because the cords are separated from first binding layer 654a by fabric layer 652.

Alternatively or additionally, such smooth sliding is facilitated by providing a friction reduced area, e.g., in the form of a pocket 650 within which spring 634 and/or cords 642 are disposed. Because pocket 650 is formed by not binding the layers of the sheet by applying heat and/or pressure, the pocket can reduce the friction between fabric layer 652 and the cords, e.g., by avoiding binding layer 654 (e.g., second binding layer 654b) binding to the cords, and/or avoiding compression of the fabric layer against the cords, frame and/or the second binding layer. Pocket 650 can be as described with reference to FIGS. 2A-3 and 7A-B, and/or can be formed using techniques described with reference to these figures.

FIG. 6C schematically illustrates an implant 600′ which can be considered to be a variant of implant 600, except that implant 600′ comprises a patch 610′ that comprises a sheet 620′ that is formed with at least one lip 606 folded over to define a hem 607, in accordance with some implementations. Sheet 620′ comprises, among other components, a frame 630 similar (e.g., identical) to the frame used in implant 600, mutatis mutandis. Implant 600′ has three lips 606, e.g., two lateral lips and one root lip, folded onto a second face of sheet 620′ (e.g., contact face 621 of the implant) thereby defining three respective hems 607 of the implant. Similar to that disclosed hereinabove with respect to FIGS. 4A to 4K, the frame can be disposed within the sheet, such that layers of the sheet can cover the frame from both sides. For example, a first coating layer 656a and a first binding layer 654a extend together beyond edges of a fabric layer 652, thereby defining lips 606. In this application, at least at some parts of patch 610′, three layers of the sheet (e.g., first coating layer 656a, first binding layer 654a and the fabric layer 652) extend beyond the lateral edges of the frame, similarly, mutatis mutandis, to as detailed hereinabove with respect to FIG. 4I. As illustrated, lip portions of first coating layer 656a and first binding layer 654a are folded over edges of fabric layer 652 (and typically the frame) onto the second face of sheet 620′, thereby forming hems 607. In the example shown, three lips 606 are folded over onto the second face of the sheet, such that the implant has three rounded edges.

In the example shown, sheet 620′ has a second coating layer 656b, on an opposite side of fabric layer 652 (and of frame 630) from coating layer 656a, and bound to the fabric layer by a second binding layer 654b that is sandwiched between the second coating layer and the fabric layer. At parts of patch 610′ in which frame 630 is disposed, the frame can be sandwiched between second binding layer 654b and fabric layer 652, e.g., as shown. Therefore, the folding of lips 606 onto the second face of sheet 620′ places the lips onto second coating layer 656b, with first binding layer 654a sandwiched between first coating layer 656a and the second coating layer, and binding together the first and second coating layers—thereby securing hems 607.

For implant 600′, the face of patch 610′ onto which lips 606 are folded can serve as a contact face 621 of the implant, e.g., a face that is placed in chronic contact with tissue.

FIG. 6D schematically illustrates an implant 600″ which can be considered to be a variant of implant 600 or variants thereof, except that implant 600″ comprises a patch 610″ that comprises a sheet 620″ that is formed with at least one lip 606″, in accordance with some implementations. Sheet 620″ comprises, among other components, a frame 630 similar (e.g., identical) to the frame used in implant 600, mutatis mutandis.

Similarly to as disclosed hereinabove with respect to FIGS. 4B-4D, 4G and 4I to 4K, frame 630 can be disposed within sheet 620″, such that layers of the sheet can cover, at least a majority of the frame from both sides. At least a majority of the frame can be covered such that at least first coating layer 656a, second coating layer 656b and first binding layer 654a of the sheet extend together beyond edges of fabric layer 652, thereby defining lips 606″. In some implementations, and as shown, sheet 620″ can include second binding layer 654b, which can also extend beyond edges of fabric layer 652 into lips 606″. In some implementations (such as that shown), lips 606″ and their formation can therefore be similar, mutatis mutandis, to lips 106″ detailed hereinabove with respect to FIG. 4K.

In some implementations, lip(s) 606″ can be softer and/or more flexible than the other portions of sheet 620″, e.g., in which fabric layer 652 and/or frame 630 are disposed. Lips 606″ may advantageously provide the implant with beneficial features similar to those described hereinabove, such as soft edges to prevent injuring tissue.

In some implementations, and as illustrated, one or more portions of frame 630 extend beyond an edge of fabric layer 652, thereby into lip 606″. In some implementations, and as illustrated, one or more portions of frame 630 protrude even beyond lip 606″, e.g., beyond an edge 601″ defined by other layers of the sheet so as to be exposed from the other layers of the sheet. In the example shown, portion 632″ of frame 630 extends beyond edge 601″.

The edge of the sheet through which the frame can protrude can be defined by any combination of the layers, e.g., the first and/or second coating layer(s), the first and/or second binding layer(s) and/or the fabric layer. In the particular example shown the frame extends into, through, and beyond a lip 606″, to protrude from edge 601″, in which the coating and binding layers, but not the fabric layer, are disposed.

Except for any portions of the frame protruding beyond edge 601″, lips 606″ can define a periphery of the sheet.

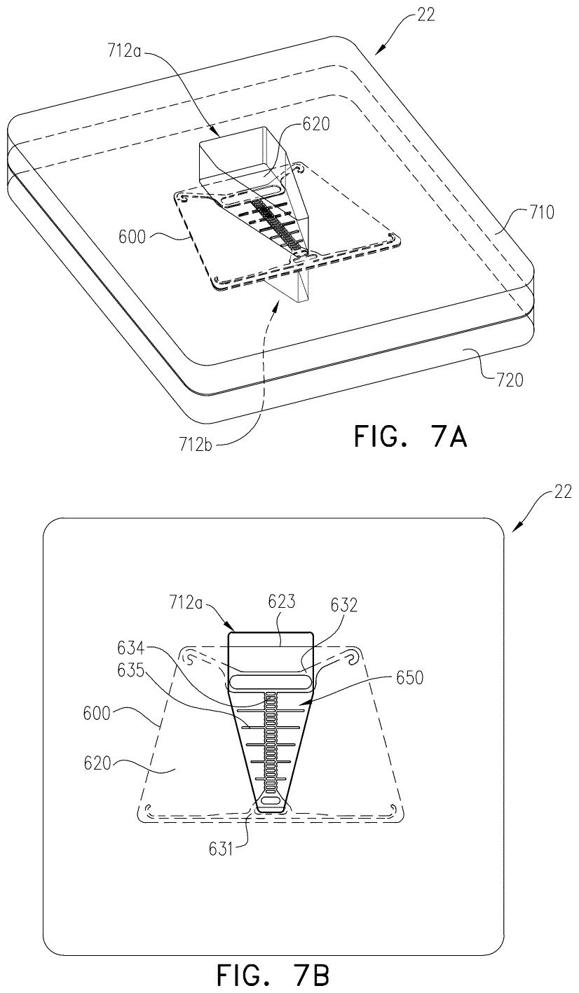

Reference is now made to FIGS. 7A and 7B, which are schematic illustrations of a laminating apparatus 22, in accordance with some implementations. The laminating apparatus comprises two plates, e.g., first plate 710 and second plate 720, configured to be pressed against each other such as to apply pressure and/or heat to a sandwich of layers (such as any one of the sandwiches of layers disclosed herein), secured between the two plates, in order to form a sheet—such as any one of the sheets disclosed herein. In the example shown, an implant 600a (which can be considered to be a variant of implant 600) is being formed with a pocket 650. In this application both plates of laminating apparatus 22 can define a recess, such as recess 712a and 712b in a contact face of the plate. The recess enables the formation of pocket 650 in the sheet (e.g., sheet 620) of the implant.

As described hereinabove, sheets can be formed by compressing and/or heating sandwiched layers, which can cause the layers to bind to each other. The pocket can be formed by reducing (e.g., eliminating) this heating and/or compression at a discrete region, which will become the region of the pocket. For example, heat and/or compression can be applied to the sandwich around the region of the pocket, but not to the actual region of the pocket. Having recess 712a and 712b at both plates enables compressing and/or applying heat to other regions of the sheet (e.g., most of the sheet) while not compressing and/or applying heat to the region designated for the pocket. Accordingly, at the region of the designated pocket, the layers do not bind—and therefore a pocket is created at that region.

When a frame is to be disposed within the sheet, the frame can be positioned between two layers of the sandwich that will become the sheet (e.g., as illustrated), prior to application of pressure and/or heat to form the sheet. The pocket can therefore be formed with at least part of the frame already disposed in the region at which the pocket will be formed. For example, and as shown, for implementations in which the recess of the plates of laminating apparatus 22 defines a recess therein, the recess can be positioned over the frame so as to form the pocket at the frame. In some implementations, part of the frame extends beyond the boundaries of the pocket.

Recess 712a and 712b can be positioned and/or shaped according to the requirements of the shape and/or position of pocket 650 and/or variants thereof. For example, in the illustrated example, recess 712a and 712b are disposed at a region (e.g., a central area) of plates 710 and 720, which in some implementations can correspond to the desired location of the pocket being at a central area of the sheet. Similarly, the shape of recess 712a and 712b is trapezoid (with a rectangular shape at the long base of the trapezoid) which will result in correspondingly-shaped pocket. When positioning the implant between the plates, recess 712 can be positioned over and/or along the central area of the frame, e.g., along spring 634 and its lateral protrusions 635, e.g., as shown. For example, the short base of the trapezoid can be at tip brace 631, whereas the long base of the trapezoid can end at root brace 632.

In some implementations, recesses 712 can extend beyond an edge of sheet 620. For example, and as shown, a rectangular part of recesses 712 can extend, from the long base of the trapezoid part of the recesses, beyond root 612 of the implant. Since the recesses extend beyond an edge of the sheet, this can result in an opening 623 being created at the edge of the sheet, e.g., as described hereinabove for opening 223. Opening 623 can enable access to cords 642, or in some implementations can enable the introduction of cords 642 subsequently to lamination. In some implementations, the pocket can be subsequently sealed, securing the frame and/or the cords therewithin—e.g., by closing the opening, such as by again heating and/or applying pressure at the opening, and/or by other means such as stitching.

In some implementations, and as illustrated, the frame is introduced into the sheet prior to the binding of the layers around the pocket, e.g., before applying heat and or pressure onto the sheet. Additionally, when forming a pocket having an opening at an edge of the sheet, a cord can extend out of the pocket via the opening (e.g., rather than out of a point 614).

Alternatively or additionally, formation of the pocket can be facilitated by an absence of a binding layer at the region that is to become the pocket, e.g., as described hereinabove with reference to FIG. 3.

Reference is now made to FIGS. 8A to 8C with are schematic illustrations of a technique for forming holes through a sheet of an implant, in accordance with some implementations. In the example shown, the technique is used to form such holes to facilitate blood flow through a wing of an implant. FIGS. 8A to 8C illustrate a sequence of at least some steps in the technique, with each of the figures showing the wing of an implant 500 from a top view (upper frame of the figures) and from a side view (lower frame of the figures). As described hereinabove, implant 500 comprises a wing that comprises sheet 550 fixedly attached to a frame 510.

In some implementations, and as can be seen in FIGS. 5 and 8A (lower figure), the wing can be curved, e.g., such that contact face 521 is concave, which may improve the implants' abilities to guide and/or provide support for the native leaflet, e.g., along the contact face, aiding the native leaflet to assume an appropriate conformation for coaptation with the opposing leaflet, as detailed in International Patent Application PCT/US2022/052834 to Chau et al., filed Dec. 14, 2022, which is incorporated herein by reference.

Cutting out accurate holes in a sheet that is already curved may be challenging compared with cutting such holes in a flat sheet, e.g., may be more time-consuming, more expensive, and/or less accurate. However, cutting out such holes from the sheet before fixedly attaching the sheet to the frame may also be challenging, e.g., with respect to aligning the sheet with the frame such that the holes are appropriately positioned with respect to the frame, e.g., avoiding a hole being covered or obstructed by a part of the frame. Furthermore, because shape-setting of frame 510 may require a temperature that sheet 550 cannot withstand, it may not be possible to confer the frame with its curvature after the sheet has been secured to the frame and the holes have been formed. Accordingly, the technique described with reference to FIGS. 8A-C advantageously facilitates formation of holes 570 subsequently to the sheet being fixedly attached to frame 510 despite the frame, at least at rest, being curved.

FIG. 8A shows sheet 550 having been fixedly attached to frame 510, prior to the formation of holes 570. At this point, implant 500 can be temporarily flattened, such that holes 570 can be formed in sheet 550 while the sheet is flat. This flattening of the implant, and/or the sheet can be facilitated by elasticity/resilience of the frame, which can enable such temporary flattening of the implant without the implant losing its curved resting shape.

FIG. 8B shows implant 500 having been temporarily flattened by a cage 800 (when positioned therewithin) having rails 810 that constrain the implant (e.g., the wing and/or the frame thereof) in a flat configuration therebetween. In some implementations, cage 800 can comprise a first set of rails 810a and a second set of rails 810b, disposed on opposing sides of cage 800, e.g., such that when cage 800 is in its closed configuration rails 810 sandwich portions of the implant therebetween (e.g., compare FIG. 8A to FIG. 8B). For example, rails 810a and 810b can be generally positioned such that in the closed configuration of the cage each rail is opposite and/or aligned with a corresponding rail disposed on the opposing side of the cage. In the particular top-view illustrations of FIGS. 8B-C, only rails 810a are visible, because rails 810b are concealed by a corresponding rail 810a disposed thereabove. In some implementations, and as can be seen, the shape of the flattened wing can be generally trapezoid, triangular, or raindrop-shaped.

FIG. 8C shows formation of holes 570 in sheet 550, while the sheet is kept in the flat configuration by rails 810 of cage 800. In some implementations, cage 800 (e.g., rails 810 thereof) is shaped to hold the implant in a flat configuration while maintaining at least a majority of sheet 550 exposed, e.g., accessible for forming the holes. The part of the wing which is exposed can be subject to formation of holes therethrough, e.g., by an automated device, or using a manual tool. Once the wing is flattened, and while it is kept flat, holes can be formed, (e.g., cut through the sheet) by laser, or mechanically (e.g., by a blade, a saw, a cutting disc, a water jet, or any other mechanical cutting machinery). For example, flattening sheet 550 can be particularly advantageous for an automated laser cutting system, e.g., such that movement of the laser head can be in a plane which can be parallel and/or coincident with a plane of the flat configuration.

In some implementations, manufacture of multiple implants can be performed by (i) attaching multiple frames in an arrangement (e.g., an array or grid) across a single sheet, (ii) flattening all of the frames concurrently (e.g., within a large cage), (iii) while constraining the frames, using an automated cutting device to form holes in the sheet at each of the frames, and (iv) subsequently, cutting the sheet to separate and/or trim the individual implants, either while the frames remain constrained, or subsequently to releasing the frames. In some implementations, the cutting of the sheet to separate and/or trim the implants can be performed using the same automated cutting machine that is used to form the holes.

In some implementations, and as shown, holes 570 are hexagonal. In some implementations, holes 570 are circular or oval.

It is to be noted that the technique described with reference to FIGS. 8A-C can be used for implants and/or sheets other than those described herein.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While the above description contains several implementations, these should not be construed as limitations on the scope of the disclosure, but rather as examples. Features of one implementation can be combined with features of other implementations. For example, features of one or more variants of implant 100 can be combined with features of one or more other variants of implant 100, features of one or more variants of implant 200 can be combined with features of one or more other variants of implant 200, and features of one or more variants of implant 100 can be combined with features of one or more variants of implant 200, mutatis mutandis. Furthermore, tools (e.g., delivery tools and/or components thereof) described for use with implant 100 (and/or variants thereof) can be used with implant 200 (and/or variants thereof), and vice versa.

### Example Implementations (Some Non-Limiting Examples of the Concepts Herein are Recited Below):

Example 1. A method for use in the manufacture of a medical implant, the method, comprising: (i) forming a sandwich that includes: (a) a fabric layer, (b) a coating layer comprising a polymer configured to inhibit tissue growth thereon, (c) a binding layer sandwiched between the fabric layer and the coating layer, and/or (d) at least part of a frame sandwiched between the fabric layer and the binding layer; and/or (ii) heating and compressing the sandwich such that the binding layer binds the fabric layer to the coating layer.

Example 2. The method according to example 1, further comprising, subsequently to heating and compressing the sandwich, sterilizing the sandwich.

Example 3. The method according to any one of examples 1-2, wherein forming the sandwich comprises sandwiching at least the part of the frame, along with the binding layer, between the fabric layer and the coating layer.

Example 4. The method according to any one of examples 1-3, wherein the coating layer consists essentially of the polymer.

Example 5. The method according to any one of examples 1-4, wherein the coating layer is nonporous.

Example 6. The method according to any one of examples 1-4, wherein the coating layer is porous.

Example 7. The method according to any one of examples 1-6, wherein the coating layer is a film.

Example 8. The method according to any one of examples 1-6, wherein the fabric layer includes a woven fabric.

Example 9. The method according to any one of examples 1-6, wherein the fabric layer includes a nonwoven fabric.

Example 10. The method according to any one of examples 1-9, wherein the polymer includes an ultra-high-molecular-weight polyethylene (UHMWPE).

Example 11. The method according to any one of examples 1-9, wherein the polymer includes a fluorinated ethylene propylene (FEP).

Example 12. The method according to any one of examples 1-11, wherein the binding layer comprises an acrylate.

Example 13. The method according to any one of examples 1-12, wherein the binding layer comprises a siloxane.

Example 14. The method according to any one of examples 1-13, wherein the binding layer comprises a thermoplastic polyurethane (TPU).

Example 15. The method according to any one of examples 1-14, wherein the binding layer is 5-30 microns thick.

Example 16. The method according to any one of examples 1-15, wherein the part of the frame is 20-500 microns thick.

Example 17. The method according to any one of examples 1-16, wherein: (i) the coating layer defines a first face of the sandwich, and the sandwich has a second face opposite to the first face, (ii) forming the sandwich comprises forming the sandwich to define a lip in which a lip-portion of the coating layer and a lip-portion of the binding layer extend together beyond an edge of the fabric layer, and/or (iii) the method further comprises folding the lip around the edge of the fabric layer onto the second face, such that the lip-portion of the binding layer is sandwiched between the lip-portion of the coating layer and the second face.

Example 18. The method according to any one of examples 1-16, wherein the frame has a first side and an opposite second side, and wherein forming the sandwich comprises wrapping the fabric layer around the frame from the first side to the second side, such that at least part of the frame is sandwiched between (i) a first part of the fabric layer disposed against the first side of the frame, and (ii) a second part of the fabric layer disposed against the second side of the frame.

Example 19. The method according to any one of examples 1-18, wherein heating and compressing the sandwich comprises heating and compressing the sandwich in a manner that defines a pocket between the fabric layer and the binding layer, with at least the part of the frame disposed in the pocket.

Example 20. The method according to example 19, wherein heating the sandwich comprises heating the sandwich between plates, at least one of the plates having a recess at a region of the pocket.

Example 21. The method according to example 19, wherein compressing the sandwich comprises compressing the sandwich between plates, at least one of the plates having a recess at a region of the pocket.

Example 22. The method according to example 19, further comprising closing the pocket by binding the fabric layer and the binding layer circumscribing the pocket.

Example 23. The method according to example 19, wherein heating and compressing the sandwich comprises heating and compressing the sandwich in a manner that provides an opening into the pocket where the fabric layer is unbound from the binding layer.

Example 24. The method according to any one of examples 1-23, wherein forming the sandwich comprises defining a lip in which a lip-portion of the coating layer and a lip-portion of the binding layer extend together beyond an edge of the fabric layer.

Example 25. The method according to example 24, wherein forming the sandwich comprises: (i) forming the sandwich such that the coating layer defines a first face of the sandwich, and such that the sandwich has a second face opposite to the first face, and/or (ii) folding the lip onto the second face such that the lip-portion of the binding layer is sandwiched between the lip-portion of the coating layer and the second face.

Example 26. The method according to any one of examples 1-25, further comprising, subsequently to heating and compressing the sandwich, forming holes through the sandwich.

Example 27. The method according to example 26, further comprising, subsequently to heating and compressing the sandwich, flattening the sandwich, and maintaining the sandwich flat while forming the holes through the sandwich.

Example 28. The method according to example 26, wherein forming the holes comprises forming the holes by laser cutting.

Example 29. The method according to example 26, wherein the holes are hexagonal holes, and wherein forming the holes comprises forming the hexagonal holes.

Example 30. A method, comprising: (i) forming a sandwich including a binding layer sandwiched between: (a) a fabric layer, and (b) a coating layer comprising a polymer configured to inhibit tissue growth thereon; and/or (ii) forming a sheet by binding the fabric layer to the coating layer by: (a) heating the sandwich, and/or (b) compressing the sandwich.

Example 31. The method according to example 1, further comprising, subsequently to heating and compressing the sandwich, sterilizing the sheet.

Example 32. The method according to any one of examples 30-31, wherein: (i) in the sandwich, the binding layer is unbound from the fabric layer, (ii) forming the sandwich comprises forming the sandwich in which the binding layer is unbound from the fabric layer, and/or (iii) forming the sheet by binding the fabric layer to the coating layer comprises forming the sheet by binding the binding layer to the fabric layer and to the coating layer by heating and compressing the sandwich.

Example 33. The method according to any one of examples 30-31, wherein: (i) in the sandwich, the binding layer is bound to the fabric layer, (ii) forming the sandwich comprises forming the sandwich in which the binding layer is bound to the fabric layer, and/or (iii) forming the sheet by binding the fabric layer to the coating layer comprises forming the sheet by binding, to the coating layer, the binding layer that is bound to the fabric layer, by heating and compressing the sandwich.

Example 34. The method according to any one of examples 30-33, wherein the coating layer consists essentially of the polymer.

Example 35. The method according to any one of examples 30-34, wherein the coating layer is nonporous.

Example 36. The method according to any one of examples 30-34, wherein the coating layer is porous.

Example 37. The method according to any one of examples 30-36, wherein the coating layer is a film.

Example 38. The method according to any one of examples 30-36, wherein the fabric layer includes a woven fabric.

Example 39. The method according to any one of examples 30-36, wherein the fabric layer includes a nonwoven fabric.

Example 40. The method according to any one of examples 30-39, wherein the polymer includes an ultra-high-molecular-weight polyethylene (UHMWPE).

Example 41. The method according to any one of examples 30-40, wherein the polymer includes a fluorinated ethylene propylene (FEP).

Example 42. The method according to any one of examples 30-41, wherein the binding layer comprises an acrylate.

Example 43. The method according to any one of examples 30-42, wherein the binding layer comprises a siloxane.

Example 44. The method according to any one of examples 30-43, wherein the binding layer comprises a thermoplastic polyurethane (TPU).

Example 45. The method according to any one of examples 30-44, wherein the fabric layer is 20-150 microns thick.

Example 46. The method according to any one of examples 30-45, wherein the binding layer is 5-30 microns thick.

Example 47. The method according to any one of examples 30-46, wherein: (i) the coating layer defines a first face of the sheet, and the sheet has a second face opposite to the first face; (ii) forming the sandwich comprises forming the sandwich to define a lip in which a lip-portion of the coating layer and a lip-portion of the binding layer extend together beyond an edge of the fabric layer, (iii) the method further comprises folding the lip around the edge of the fabric layer onto the second face, such that the lip-portion of the binding layer is sandwiched between the lip-portion of the coating layer and the second face; and/or (iv) forming the sheet comprises forming the sheet by: (a) heating the sandwich comprising the lip folded around the edge of the fabric layer onto the second face, and/or (b) compressing the sandwich comprising the lip folded around the edge of the fabric layer onto the second face.

Example 48. The method according to any one of examples 30-47, wherein forming the sheet comprises forming the sheet by binding the binding layer to the fabric layer by heating and compressing the binding layer against the fabric layer.

Example 49. The method according to example 48, wherein forming the sheet comprises forming the sheet by binding the coating layer to the binding layer by heating and compressing the coating layer against the binding layer.

Example 50. The method according to any one of examples 30-49, wherein forming the sandwich comprises defining a lip in which a lip-portion of the coating layer and a lip-portion of the binding layer extend together beyond an edge of the fabric layer.

Example 51. The method according to example 50, wherein forming the sandwich comprises: (i) forming the sandwich such that the coating layer defines a first face of the sheet, and such that the sheet has a second face opposite to the first face, and/or (ii) folding the lip onto the second face such that the lip-portion of the binding layer is sandwiched between the lip-portion of the coating layer and the second face.

Example 52. The method according to any one of examples 30-51, wherein: (i) the sandwich includes at least a part of a frame sandwiched between the fabric layer and the binding layer, and/or (ii) forming the sheet comprises forming the sheet by binding the fabric layer to the coating layer, with at least the part of the frame disposed between the fabric layer and the binding layer, by: (a) heating the sandwich that includes at least the part of the frame sandwiched between the fabric layer and the binding layer, and/or (b) compressing the sandwich that includes at least the part of the frame sandwiched between the fabric layer and the binding layer.

Example 53. The method according to example 52, wherein forming the sheet comprises forming the sheet to define a pocket between the fabric layer and the binding layer by heating and compressing the sandwich around a region of the pocket, such that at least part of the frame is disposed in the pocket.

Example 54. The method according to example 53, wherein heating the sandwich comprises heating the sandwich between plates, at least one of the plates having a recess at the region of the pocket.

Example 55. The method according to example 53, wherein compressing the sandwich comprises compressing the sandwich between plates, at least one of the plates having a recess at the region of the pocket.

Example 56. The method according to example 52, wherein forming the sheet comprises forming the sheet by binding the fabric layer to the coating layer in a manner that forms a pocket between the fabric layer and the binding layer.

Example 57. The method according to example 56, wherein forming the pocket comprises forming the pocket by: (i) heating the sandwich around an area of the pocket, and/or (ii) compressing the sandwich around the area of the pocket.

Example 58. The method according to example 56, further closing the pocket by binding the fabric layer and the binding layer circumscribing the pocket.

Example 59. The method according to example 53, wherein forming the sheet comprises forming the sheet having an opening at an edge of the sheet by having the fabric layer unbound from the binding layer.

Example 60. The method according to example 52, wherein forming the sandwich comprises sandwiching at least the part of the frame, along with the binding layer, between the fabric layer and the coating layer.

Example 61. The method according to example 60, wherein a thickness of the frame between the fabric layer and the binding layer is 20-500 microns.

Example 62. The method according to any one of examples 30-61, wherein the method further comprises covering at least part of a frame by fixedly attaching the sheet to the frame.

Example 63. The method according to example 62, wherein fixedly attaching the sheet to the frame comprises stitching the sheet to the frame.

Example 64. The method according to any one of examples 30-63, wherein forming the sandwich comprises forming the sandwich including a frame sandwiched, along with the binding layer, between the fabric layer and the coating layer.

Example 65. The method according to example 64, wherein a thickness of the frame between the fabric layer and the binding layer is 20-500 microns.

Example 66. The method according to example 64, wherein the frame has a first side and an opposite second side, and wherein forming the sandwich comprises wrapping the fabric layer around the frame from the first side to the second side, such that at least part of the frame is sandwiched between (i) a first part of the fabric layer disposed against the first side of the frame, and (ii) a second part of the fabric layer disposed against the second side of the frame.

Example 67. The method according to example 62, further comprising forming multiple holes through the sheet, to facilitate blood flow therethrough.

Example 68. The method according to example 67, further comprising flattening the sheet and keeping it flat while forming the multiple holes through the sheet.

Example 69. The method according to example 67, wherein forming the holes comprises forming the holes by laser cutting.

Example 70. The method according to example 67, wherein each of the holes is hexagonal.

Example 71. The method according to any one of examples 30-70, wherein: (i) the binding layer is a first binding layer, (ii) the coating layer is a first coating layer, (iii) forming the sandwich comprises forming the sandwich to include a second binding layer sandwiched between the fabric layer and a second coating layer configured to inhibit tissue growth thereon; and/or (iv) forming the sheet comprises binding the fabric layer to the second coating layer by heating and compressing the sandwich.

Example 72. The method according to example 71, wherein: (i) the coating layer defines a first face of the sheet, and the sheet has a second face opposite to the first face; (ii) forming the sandwich comprises forming the sandwich to define a lip in which a lip-portion of the coating layer and a lip-portion of the binding layer extend together beyond an edge of the fabric layer; (iii) the method further comprises folding the lip around the edge of the fabric layer onto the second face, such that the lip-portion of the binding layer is sandwiched between the lip-portion of the coating layer and the second face; and/or (iv) forming the sheet comprises forming the sheet by: (a) heating the sandwich comprising the lip folded around the edge of the fabric layer onto the second face, and/or (b) compressing the sandwich comprising the lip folded around the edge of the fabric layer onto the second face.

Example 73. The method according to example 71, wherein the first coating layer, the second coating layer, and the first binding layer extend together beyond an edge of the fabric layer, thereby defining a lip, and forming the sandwich including the lip in which a lip-portion of the first coating layer, a lip-portion of the second coating layer and a lip-portion of the first binding layer are disposed.

Example 74. The method according to example 73, wherein forming the sandwich comprises: (i) forming the sandwich such that the coating layer defines a first face of the sheet, and such that the sheet has a second face opposite to the first face, and/or (ii) folding the lip onto the second face such that the lip-portion of the binding layer is sandwiched between the lip-portion of the coating layer and the second face.

Example 75. The method according to example 71, wherein the first coating layer, the second coating layer, the first binding layer and the second binding layer extend together beyond an edge of the fabric layer, thereby defining a lip, and forming the sandwich including the lip in which a lip-portion of the first coating layer, a lip-portion of the second coating layer and a lip-portion of the first binding layer are disposed.

Example 76. The method according to any one of examples 30-75, wherein forming the sheet comprises forming the sheet by binding the fabric layer to the coating layer in a manner that forms a pocket between the fabric layer and the binding layer.

Example 77. The method according to example 76, wherein forming the sheet in the manner that forms the pocket comprises: (i) heating the sandwich around an area of the pocket, and/or (ii) compressing the sandwich around the area of the pocket.

Example 78. The method according to example 77, wherein heating the sandwich around the area of the pocket comprises positioning the sandwich between plates, at least one of the plates being heated, and at least one of the plates having a recess that defines the area of the pocket.

Example 79. The method according to example 77, wherein compressing the sandwich around the area of the pocket comprises compressing the sandwich between plates, at least one of the plates having a recess that defines the area of the pocket.

Example 80. The method according to example 76, further comprising closing the pocket by binding between the fabric layer and the binding layer circumscribing the pocket.

Example 81. The method according to example 80, wherein the sheet defines an opening into the pocket.

Example 82. The method according to example 81, wherein, at the opening, the fabric layer is unbound from the binding layer.

Example 83. The method according to example 81, wherein the opening is disposed at an edge the sheet.

Example 84. The method according to example 76, further comprising a cord, at least a portion of the cord extending through the pocket.

Example 85. The method according to example 84, wherein the cord is slidable within the pocket.

Example 86. The method according to any one of examples 30-85, wherein the polymer is a polytetrafluoroethylene (PTFE).

Example 87. The method according to example 86, wherein the polymer is an expanded PTFE (ePTFE).

Example 88. The method according to example 86, wherein the coating layer comprises several layered sublayers of the polymer.

Example 89. The method according to example 88, wherein each of the sublayers is anisotropic, and wherein the sublayers are arranged such that each of the sublayers is disposed orthogonally to an adjacent one of the sublayers.

Example 90. The method according to example 88, wherein the coating layer comprises less than ten of the sublayers.

Example 91. The method according to example 90, wherein the coating layer comprises less than six of the sublayers.

Example 92. The method according to any one of examples 30-91, wherein compressing the sandwich comprises compressing the sandwich at a pressure of Example 0.01-1 MPa.

Example 93. The method according to example 92, wherein compressing the sandwich comprises holding the sandwich at the pressure for 100-500 seconds.

Example 94. The method according to any one of examples 30-93, wherein heating the sandwich comprises heating the sandwich to a temperature of 37-260 degrees Celsius.

Example 95. The method according to example 94, wherein heating the sandwich comprises holding the sandwich at the temperature for 100-500 seconds.

Example 96. A system and/or an apparatus, comprising a sheet that comprises: (i) a fabric layer; (ii) a coating layer comprising a polymer configured to inhibit tissue growth thereon; and/or (iii) a binding layer sandwiched between the fabric layer and the coating layer, and binding the fabric layer to the coating layer.

Example 97. The system/apparatus according to example 96, wherein the system/apparatus is shaped to define a leaflet-augmentation patch, configured to be attached to a leaflet of a valve of a heart of a subject.

Example 98. The system/apparatus according to any one of examples 96-97, wherein the coating layer consists essentially of the polymer.

Example 99. The system/apparatus according to any one of examples 96-98, wherein the coating layer is nonporous.

Example 100. The system/apparatus according to any one of examples 96-98, wherein the coating layer is porous.

Example 101. The system/apparatus according to any one of examples 96-100, wherein the coating layer is a film.

Example 102. The system/apparatus according to any one of examples 96-100, wherein the fabric layer comprises is a woven fabric.

Example 103. The system/apparatus according to any one of examples 96-100, wherein the fabric layer comprises a nonwoven fabric.

Example 104. The system/apparatus according to any one of examples 96-103, wherein the polymer is an ultra-high-molecular-weight polyethylene (UHMWPE).

Example 105. The system/apparatus according to any one of examples 96-104, wherein the polymer is a fluorinated ethylene propylene (FEP).

Example 106. The system/apparatus according to any one of examples 96-105, wherein the binding layer comprises an acrylate.

Example 107. The system/apparatus according to any one of examples 96-106, wherein the binding layer comprises a siloxane.

Example 108. The system/apparatus according to any one of examples 96-107, wherein the fabric layer is 20-150 microns thick.

Example 109. The system/apparatus according to any one of examples 96-108, wherein the binding layer is 5-30 microns thick.

Example 110. The system/apparatus according to any one of examples 96-109, wherein the coating layer and the binding layer extend together beyond an edge of the fabric layer, thereby defining a lip in which a lip-portion of the coating layer and a lip-portion of the binding layer are disposed.

Example 111. The system/apparatus according to example 110, wherein the coating layer defines a first face of the sheet, and the sheet has a second face opposite to the first face, and wherein the lip is folded onto the second face, such that the lip-portion of the binding layer is sandwiched between the lip-portion of the coating layer and the second face.

Example 112. The system/apparatus according to example 110, wherein the lip is folded around the edge of the fabric layer, thereby defining a hem of the sheet within which the fabric layer is disposed between the lip-portion of the binding layer and another portion of the binding layer.

Example 113. The system/apparatus according to any one of examples 96-112, wherein the sheet defines a pocket between the fabric layer and the binding layer.

Example 114. The system/apparatus according to example 113, wherein the binding between the fabric layer and the binding layer circumscribes the pocket, thereby closing the pocket.

Example 115. The system/apparatus according to example 113, wherein the sheet defines an opening into the pocket.

Example 116. The system/apparatus according to example 115, wherein, at the opening, the fabric layer is unbound from the binding layer.

Example 117. The system/apparatus according to example 115, wherein the opening is disposed at an edge of the sheet.

Example 118. The system/apparatus according to example 113, further comprising a cord, at least a portion of the cord extending through the pocket.

Example 119. The system/apparatus according to example 118, wherein the portion of the cord is slidable within the pocket.

Example 120. The system/apparatus according to any one of examples 96-119, wherein the system/apparatus is shaped as a wing defining a contact face, and an opposing face opposite to the contact face.

Example 121. The system/apparatus according to example 120, wherein the wing has multiple holes configured to facilitate blood flow therethrough.

Example 122. The system/apparatus according to example 121, wherein each of the holes is hexagonal.

Example 123. The system/apparatus according to example 120, wherein the wing configured to be anchored to a tissue of a heart of a subject having a first leaflet and a second opposing leaflet, such that the wing extends over the first leaflet toward the second leaflet, and the contact face faces the first leaflet.

Example 124. The system/apparatus according to any one of examples 96-123, further comprising a frame, the sheet being fixedly attached to the frame in a manner that covers at least part of the frame.

Example 125. The system/apparatus according to example 124, wherein the sheet is fixedly attached to the frame by stitching.

Example 126. The system/apparatus according to example 124, wherein the sheet is fixedly attached to the frame by adhesive.

Example 127. The system/apparatus according to example 124, wherein the sheet is fixedly attached to the frame by the frame being sandwiched between the fabric layer and the binding layer.

Example 128. The system/apparatus according to example 127, wherein: (i) the coating layer is a first coating layer, (ii) the binding layer is a first binding layer, and/or (iii) the sheet further comprises: (a) a second coating layer, and/or (b) a second binding layer, sandwiched between the frame and the second coating layer.

Example 129. The system/apparatus according to example 127, wherein a thickness of the frame between the fabric layer and the binding layer is 20-500 microns.

Example 130. The system/apparatus according to example 127, wherein the frame has a first side and an opposite second side, and the fabric layer is wrapped around from the first side to the second side, such that at least part of the frame is sandwiched between (i) a first part of the fabric layer disposed against the first side of the frame, and (ii) a second part of the fabric layer disposed against the second side of the frame.

Example 131. The system/apparatus according to example 130, wherein: (i) the coating layer is a first coating layer, (ii) the binding layer is a first binding layer, and/or (iii) the sheet further comprises: (a) a second coating layer, and/or (b) a second binding layer, sandwiched between the second part of the fabric layer and the second coating layer.

Example 132. The system/apparatus according to example 124, wherein the frame is flexible, and the system/apparatus defines a wing having a contact face, and an opposing face opposite to the contact face.

Example 133. The system/apparatus according to example 132, wherein the sheet has multiple holes configured to facilitate blood flow therethrough.

Example 134. The system/apparatus according to example 132, wherein the wing is configured to be anchored to an annulus of a valve of a heart of a subject such that the wing extends over a first leaflet of the valve toward an opposing leaflet of the valve, and the contact face faces the first leaflet.

Example 135. The system/apparatus according to example 124, wherein the system/apparatus has a pocket between the fabric layer and the binding layer, at least part of the frame being disposed in the pocket.

Example 136. The system/apparatus according to example 124, wherein the coating layer and the binding layer extend together beyond an edge of the frame, thereby defining a lip in which a lip-portion of the coating layer and a lip-portion of the binding layer are disposed.

Example 137. The system/apparatus according to example 136, wherein the frame is disposed between the fabric layer and the binding layer, and wherein the fabric layer extends, together with the coating layer and the binding layer, beyond the edge of the frame.

Example 138. The system/apparatus according to example 136, wherein the coating layer and the binding layer extend together beyond an edge of the fabric layer, thereby defining the lip.

Example 139. The system/apparatus according to example 136, wherein the coating layer defines a first face of the sheet, and the sheet has a second face opposite to the first face, and wherein the lip is folded onto the second face, such that the lip-portion of the binding layer is disposed between the lip-portion of the coating layer and the second face.

Example 140. The system/apparatus according to example 139, wherein at least part of the frame is disposed between the lip-portion of the binding layer and another portion of the binding layer.

Example 141. The system/apparatus according to example 136, wherein the lip is folded around the edge of the frame, thereby defining a hem of the sheet within which the edge of the frame is disposed between the lip-portion of the binding layer and another portion of the binding layer.

Example 142. The system/apparatus according to example 141, wherein at least a part of the fabric layer is disposed within the hem.

Example 143. The system/apparatus according to any one of examples 96-142, wherein the coating layer is a first coating layer, the binding layer is a first binding layer, and the sheet further comprises: (i) a second coating layer; and/or (ii) a second binding layer, sandwiched between the fabric layer and the second coating layer, and binding the fabric layer to the second coating layer, the fabric layer sandwiched between the first binding layer and the second binding layer.

Example 144. The system/apparatus according to example 143, wherein the sheet defines a pocket between the fabric layer and the first binding layer.

Example 145. The system/apparatus according to example 143, wherein the first coating layer, the second coating layer, and the first binding layer extend together beyond an edge of the fabric layer, thereby defining a lip in which a lip-portion of the first coating layer, a lip-portion of the second coating layer, and a lip-portion of the first binding layer are disposed.

Example 146. The system/apparatus according to example 145, wherein, within the lip, the lip-portion of the first coating layer is bound to the lip-portion of the second coating layer by the lip-portion of the first binding layer.

Example 147. The system/apparatus according to example 145, wherein a lip-portion of the second binding layer extends beyond the edge of the fabric layer into the lip such that, within the lip, the lip-portion of the first coating layer is bound to the lip-portion of the second coating layer by the lip-portion of the first binding layer and the lip-portion of the second binding layer.

Example 148. The system/apparatus according to example 145, further comprising a frame, disposed between the first and the second binding layers.

Example 149. The system/apparatus according to example 148, wherein, at at least part of the sheet, the lip extends beyond an edge of the frame.

Example 150. The system/apparatus according to example 148, wherein, at least part of the frame extends beyond an edge of the lip.

Example 151. The system/apparatus according to example 143, wherein the first coating layer and the first binding layer extend together beyond an edge of the fabric layer, thereby defining a lip in which a lip-portion of the first coating layer and a lip-portion of the first binding layer are disposed.

Example 152. The system/apparatus according to example 151, wherein the first coating layer defines a first face of the sheet, and the sheet has a second face opposite to the first face, and wherein the lip is folded onto the second face such that the lip-portion of the first binding layer is disposed between the lip-portion of the first coating layer and the second face.

Example 153. The system/apparatus according to example 152, wherein the lip is folded onto the second face such that the lip-portion of the first binding layer is disposed between the lip-portion of the first coating layer and the second coating layer.

Example 154. The system/apparatus according to example 143, further comprising a frame, the sheet being fixedly attached to the frame in a manner that covers at least part of the frame, wherein: (i) the first coating layer defines a first face of the sheet, and the sheet has a second face opposite to the first face, and/or (ii) a lip-portion of the first coating layer and a lip-portion of the first binding layer extend together beyond an edge of the fabric layer, thereby defining a lip, the lip extending beyond an edge of the frame.

Example 155. The system/apparatus according to example 154, wherein the lip is folded onto the second face, thereby defining a hem of the sheet within which at least a part of the frame is disposed.

Example 156. The system/apparatus according to example 154, wherein a lip-portion of the second coating layer and a lip-portion of the second binding layer extend together beyond an edge of the fabric layer, into the lip.

Example 157. The system/apparatus according to example 143, further comprising a frame, the sheet being fixedly attached to the frame in a manner that covers at least part of the frame.

Example 158. The system/apparatus according to example 157, wherein the system/apparatus has a pocket disposed between the fabric layer and the first binding layer, at least part of the frame being disposed in the pocket, between the fabric layer and the first binding layer.

Example 159. The system/apparatus according to example 158, wherein the pocket is closed by the binding between the fabric layer and the first binding layer circumscribing the pocket.

Example 160. The system/apparatus according to example 158, wherein the sheet defines an opening into the pocket.

Example 161. The system/apparatus according to example 160, wherein, at the opening, the fabric layer is unbound from the first binding layer.

Example 162. The system/apparatus according to example 160, wherein the opening is disposed at an edge of the sheet.

Example 163. The system/apparatus according to example 158, further comprising a cord, attached to the frame, at least a portion of the cord extending through the pocket.

Example 164. The system/apparatus according to example 163, wherein the portion of the cord is slidable within the pocket.

Example 165. The system/apparatus according to any one of examples 96-164, wherein the polymer is a polytetrafluoroethylene (PTFE).

Example 166. The system/apparatus according to example 165, wherein the polymer is an expanded PTFE (ePTFE).

Example 167. The system/apparatus according to example 165, wherein the coating layer comprises several layered sublayers of the polymer.

Example 168. The system/apparatus according to example 167, wherein each of the sublayers is anisotropic, and wherein the sublayers are arranged such that each of the sublayers is disposed orthogonally to an adjacent one of the sublayers.

Example 169. The system/apparatus according to example 167, wherein the coating layer comprises less than ten of the sublayers.

Example 170. The system/apparatus according to example 169, wherein the coating layer comprises less than six of the sublayers.

Example 171. The system/apparatus according to any one of examples 96-170, wherein the binding layer comprises a thermoplastic polyurethane (TPU).

Example 172. The system/apparatus according to example 171, wherein the TPU is a polycarbonate-based TPU.

Example 173. The system/apparatus according to example 172, wherein the polycarbonate-based TPU is a polycarbonate-based aliphatic thermoplastic polyurethane.

Example 174. The system/apparatus according to any one of examples 96-173, wherein the sheet is formed by binding the binding layer to the fabric layer and the coating layer by compressing together and heating the fabric layer, the coating layer, and the binding layer.

Example 175. The system/apparatus according to example 174, wherein the sheet is formed by compressing together the fabric layer, the coating layer, and the binding layer for 100-500 seconds.

Example 176. The system/apparatus according to example 174, wherein the sheet is formed by compressing together the fabric layer, the coating layer, and the binding layer for 20-420 seconds.

Example 177. The system/apparatus according to example 174, wherein the sheet is formed by heating the fabric layer, the coating layer, and the binding layer for 100-500 seconds.

Example 178. The system/apparatus according to example 174, wherein the sheet is formed by heating the fabric layer, the coating layer, and the binding layer for 20-420 seconds.

Example 179. The system/apparatus according to example 174, wherein the sheet is formed by compressing together the fabric layer, the coating layer, and the binding layer at a pressure of Example 0.01-1 MPa.

Example 180. The system/apparatus according to example 179, wherein the sheet is formed by compressing together the fabric layer, the coating layer, and the binding layer at the pressure for 100-500 seconds.

Example 181. The system/apparatus according to example 179, wherein the sheet is formed by compressing together the fabric layer, the coating layer, and the binding layer at the pressure for 20-420 seconds.

Example 182. The system/apparatus according to example 174, wherein the sheet is formed by heating the fabric layer, the coating layer, and the binding layer to a temperature of 37-260 degrees Celsius.

Example 183. The system/apparatus according to example 182, wherein the sheet is formed by holding the fabric layer, the coating layer, and the binding layer at the temperature for 100-500 seconds.

Example 184. The system/apparatus according to example 182, wherein the sheet is formed by holding the fabric layer, the coating layer, and the binding layer at the temperature for 20-420 seconds.

Example 185. A system and/or an apparatus, comprising a cardiovascular implant that comprises: (ii) a frame; and/or (ii) a sheet, comprising: (a) a fabric layer secured against at least a portion of the frame; (b) a coating layer, comprising a polymer configured to inhibit tissue growth thereon; and/or (c) a binding layer, sandwiched between the fabric layer and the coating layer, and binding the fabric layer to the coating layer.

Example 186. The system/apparatus according to example 185, wherein the portion of the frame is a first portion of the frame, and wherein a second portion of the frame is exposed from the sheet.

Example 187. The system/apparatus according to any one of examples 185-186, wherein the frame is designed as a frame for a prosthetic valve.

Example 188. The system/apparatus according to any one of examples 185-187, wherein the frame is designed as a leaflet-augmentation patch.

Example 189. The system/apparatus according to any one of examples 185-188, wherein the frame is designed as a wing.

Example 190. The system/apparatus according to any one of examples 185-187, wherein the frame is designed as a frame for a stent.

Example 191. The system/apparatus according to any one of examples 185-190, wherein the implant is configured to be anchored to a tissue of a heart of a subject.

Example 192. The system/apparatus according to any one of examples 185-191, wherein the sheet is fixedly attached to the frame by stitching.

Example 193. The system/apparatus according to any one of examples 185-192, wherein the sheet is fixedly attached to the frame by adhesive.

Example 194. The system/apparatus according to any one of examples 185-193, wherein the system/apparatus has a pocket between the fabric layer and the binding layer, at least part of the frame being disposed in the pocket.

Example 195. The system/apparatus according to any one of examples 185-194, wherein the coating layer consists essentially of the polymer.

Example 196. The system/apparatus according to any one of examples 185-195, wherein the coating layer is nonporous.

Example 197. The system/apparatus according to any one of examples 185-195, wherein the coating layer is porous.

Example 198. The system/apparatus according to any one of examples 185-197, wherein the coating layer is a film.

Example 199. The system/apparatus according to any one of examples 185-197, wherein the fabric layer comprises a woven fabric.

Example 200. The system/apparatus according to any one of examples 185-197, wherein the fabric layer comprises a nonwoven fabric.

Example 201. The system/apparatus according to any one of examples 185-200, wherein the polymer is an ultra-high-molecular-weight polyethylene (UHMWPE).

Example 202. The system/apparatus according to any one of examples 185-201, wherein the polymer is a fluorinated ethylene propylene (FEP).

Example 203. The system/apparatus according to any one of examples 185-202, wherein the binding layer comprises an acrylate.

Example 204. The system/apparatus according to any one of examples 185-203, wherein the binding layer comprises a siloxane.

Example 205. The system/apparatus according to any one of examples 185-204, wherein the fabric layer is 20-150 microns thick.

Example 206. The system/apparatus according to any one of examples 185-205, wherein the binding layer is 5-30 microns thick.

Example 207. The system/apparatus according to any one of examples 185-206, wherein the coating layer and the binding layer extend together beyond an edge of the frame, thereby defining a lip in which a lip-portion of the coating layer and a lip-portion of the binding layer are disposed.

Example 208. The system/apparatus according to example 207, wherein the frame is disposed between the fabric layer and the binding layer, and wherein the fabric layer extends, together with the coating layer and the binding layer, beyond the edge of the frame.

Example 209. The system/apparatus according to example 207, wherein the coating layer and the binding layer extend together beyond an edge of the fabric layer, thereby defining the lip.

Example 210. The system/apparatus according to example 207, wherein the coating layer defines a first face of the sheet, and the sheet has a second face opposite to the first face, and wherein the lip is folded onto the second face, such that the lip-portion of the binding layer is disposed between the lip-portion of the coating layer and the second face.

Example 211. The system/apparatus according to example 210, wherein at least part of the frame is disposed between the lip-portion of the binding layer and another portion of the binding layer.

Example 212. The system/apparatus according to example 210, wherein the lip is folded around the edge of the frame, thereby defining a hem of the sheet within which the edge of the frame is disposed between the lip-portion of the binding layer and another portion of the binding layer.

Example 213. The system/apparatus according to example 212, wherein at least a part of the fabric layer is disposed within the hem.

Example 214. The system/apparatus according to any one of examples 185-213, wherein the fabric layer is secured against at least the portion of the frame by at least the portion of the frame being sandwiched between the binding layer and the fabric layer.

Example 215. The system/apparatus according to example 214, wherein the portion of the frame is a first portion of the frame, and wherein a second portion of the frame is disposed within a pocket between the binding layer and the fabric layer.

Example 216. The system/apparatus according to any one of examples 185-215, wherein the sheet is fixedly attached to the frame by the frame being sandwiched between the fabric layer and the binding layer.

Example 217. The system/apparatus according to example 216, wherein a thickness of the frame between the fabric layer and the binding layer is 20-500 microns.

Example 218. The system/apparatus according to example 216, wherein the frame has a first side and an opposite second side, and the fabric layer is wrapped around from the first side to the second side, such that at least part of the frame is sandwiched between (i) a first part of the fabric layer disposed against the first side of the frame, and (ii) a second part of the fabric layer disposed against the second side of the frame.

Example 219. The system/apparatus according to example 216, wherein: (i) the coating layer is a first coating layer, (ii) the binding layer is a first binding layer, and/or (iii) the sheet further comprises: (a) a second coating layer, and/or (b) a second binding layer, sandwiched between the fabric layer and the second coating layer.

Example 220. The system/apparatus according to any one of examples 185-219, wherein the frame is flexible, and the implant defines a wing having a contact face, and an opposing face opposite to the contact face.

Example 221. The system/apparatus according to example 220, wherein the sheet has multiple holes configured to facilitate blood flow therethrough.

Example 222. The system/apparatus according to example 220, wherein the wing is configured to be anchored to an annulus of a valve of a heart of a subject such that the wing extends over a first leaflet of the valve toward an opposing leaflet of the valve, and the contact face faces the first leaflet.

Example 223. The system/apparatus according to any one of examples 185-222, wherein the coating layer is a first coating layer, the binding layer is a first binding layer, and the sheet further comprises: (i) a second coating layer; and/or (ii) a second binding layer, sandwiched between the fabric layer and the second coating layer, and binding the fabric layer to the second coating layer, the fabric layer sandwiched between the first binding layer and the second binding layer.

Example 224. The system/apparatus according to example 223, wherein the sheet defines a pocket between the fabric layer and the first binding layer.

Example 225. The system/apparatus according to example 223, wherein the first coating layer and the first binding layer extend together beyond an edge of the fabric layer, thereby defining a lip in which a lip-portion of the first coating layer and a lip-portion of the first binding layer are disposed.

Example 226. The system/apparatus according to example 225, wherein the first coating layer defines a first face of the sheet, and the sheet has a second face opposite to the first face, and wherein the lip is folded onto the second face such that the lip-portion of the first binding layer is disposed between the lip-portion of the first coating layer and the second face.

Example 227. The system/apparatus according to example 226, wherein the lip is folded onto the second face such that the lip-portion of the first binding layer is disposed between the lip-portion of the first coating layer and the second coating layer.

Example 228. The system/apparatus according to example 223, wherein the first coating layer, the second coating layer, and the first binding layer extend together beyond an edge of the fabric layer, thereby defining a lip in which a lip-portion of the first coating layer, a lip-portion of the second coating layer, and a lip-portion of the first binding layer are disposed.

Example 229. The system/apparatus according to example 228, wherein, within the lip, the lip-portion of the first coating layer is bound to the lip-portion of the second coating layer by the lip-portion of the first binding layer.

Example 230. The system/apparatus according to example 228, wherein a lip-portion of the second binding layer extends beyond the edge of the fabric layer into the lip such that, within the lip, the lip-portion of the first coating layer is bound to the lip-portion of the second coating layer by the lip-portion of the first binding layer and the lip-portion of the second binding layer.

Example 231. The system/apparatus according to example 228, wherein, at at least part of the sheet, the lip extends beyond an edge of the frame.

Example 232. The system/apparatus according to example 228, wherein, at least part of the frame extends beyond an edge of the lip.

Example 233. The system/apparatus according to example 228, wherein the lip is folded to define a hem of the sheet within which at least a part of the frame is disposed.

Example 234. The system/apparatus according to example 228, wherein a lip-portion of the second coating layer and a lip-portion of the second binding layer extend together beyond an edge of the fabric layer, into the lip.

Example 235. The system/apparatus according to example 223, wherein the sheet is fixedly attached to the frame in a manner that covers at least part of the frame.

Example 236. The system/apparatus according to example 235, wherein the system/apparatus has a pocket disposed between the fabric layer and the first binding layer, at least part of the frame being disposed in the pocket, between the fabric layer and the first binding layer.

Example 237. The system/apparatus according to example 236, wherein the pocket is closed by the binding between the fabric layer and the first binding layer circumscribing the pocket.

Example 238. The system/apparatus according to example 236, wherein the sheet defines an opening into the pocket.

Example 239. The system/apparatus according to example 238, wherein, at the opening, the fabric layer is unbound from the first binding layer.

Example 240. The system/apparatus according to example 238, wherein the opening is disposed at an edge of the sheet.

Example 241. The system/apparatus according to example 238, further comprising a cord, attached to the frame, at least a portion of the cord extending through the pocket.

Example 242. The system/apparatus according to example 241, wherein the portion of the cord is slidable within the pocket.

Example 243. The system/apparatus according to any one of examples 185-242, wherein the polymer is a polytetrafluoroethylene (PTFE).

Example 244. The system/apparatus according to example 243, wherein the polymer is an expanded PTFE (ePTFE).

Example 245. The system/apparatus according to example 243, wherein the coating layer comprises several layered sublayers of the polymer.

Example 246. The system/apparatus according to example 245, wherein each of the sublayers is anisotropic, and wherein the sublayers are arranged such that each of the sublayers is disposed orthogonally to an adjacent one of the sublayers.

Example 247. The system/apparatus according to example 245, wherein the coating layer comprises less than ten of the sublayers.

Example 248. The system/apparatus according to example 247, wherein the coating layer comprises less than six of the sublayers.

Example 249. The system/apparatus according to any one of examples 185-248, wherein the binding layer comprises a thermoplastic polyurethane (TPU).

Example 250. The system/apparatus according to example 249, wherein the TPU is a polycarbonate-based TPU.

Example 251. The system/apparatus according to example 250, wherein the polycarbonate-based TPU is a polycarbonate-based aliphatic thermoplastic polyurethane.

Example 252. The system/apparatus according to any one of examples 185-251, wherein the sheet is formed by binding the binding layer to the fabric layer and the coating layer by compressing together and heating the fabric layer, the coating layer, and the binding layer.

Example 253. The system/apparatus according to example 252, wherein the sheet is formed by heating the fabric layer, the coating layer, and the binding layer for 100-500 seconds.

Example 254. The system/apparatus according to example 252, wherein the sheet is formed by heating the fabric layer, the coating layer, and the binding layer for 20-420 seconds.

Example 255. The system/apparatus according to example 252, wherein the sheet is formed by compressing together the fabric layer, the coating layer, and the binding layer for 100-500 seconds.

Example 256. The system/apparatus according to example 252, wherein the sheet is formed by compressing together the fabric layer, the coating layer, and the binding layer for 20-420 seconds.

Example 257. The system/apparatus according to example 252, wherein the sheet is formed by compressing together the fabric layer, the coating layer, and the binding layer at a pressure of Example 0.01-1 MPa.

Example 258. The system/apparatus according to example 257, wherein the sheet is formed by compressing together the fabric layer, the coating layer, and the binding layer at the pressure for 100-500 seconds.

Example 259. The system/apparatus according to example 257, wherein the sheet is formed by compressing together the fabric layer, the coating layer, and the binding layer at the pressure for 20-420 seconds.

Example 260. The system/apparatus according to example 252, wherein the sheet is formed by heating the fabric layer, the coating layer, and the binding layer to a temperature of 37-260 degrees Celsius.

Example 261. The system/apparatus according to example 260, wherein the sheet is formed by holding the fabric layer, the coating layer, and the binding layer at the temperature for 100-500 seconds.

Example 262. The system/apparatus according to example 260, wherein the sheet is formed by holding the fabric layer, the coating layer, and the binding layer at the temperature for 20-420 seconds.

Example 263. A method, comprising: (i) temporarily flattening a sheet of an implant by constraining a frame of the implant, the sheet being fixedly attached to the frame; (ii) forming holes in the sheet while the sheet remains flattened; and/or (iii) subsequently, allowing the frame to unflatten toward a resting shape in which the frame holds the sheet in a curved configuration.

Example 264. The method according to example 263, wherein forming the holes comprises forming the holes using an automated laser cutting system.

Example 265. The method according to any one of examples 263-264, wherein forming the holes comprises forming the holes by moving a laser head of a laser cutting system along a plane parallel to the flattened sheet.

Example 266. The method according to any one of examples 263-265, wherein flattening the sheet comprises flattening the sheet using a cage having rails configured to hold the sheet flattened.

Example 267. The method according to any one of examples 263-266, further comprising forming the sheet by binding a fabric layer to a coating layer by: (i) forming a sandwich including a binding layer sandwiched between the fabric layer and the coating layer; (ii) heating the sandwich, and/or (iii) compressing the sandwich.

Example 268. The method according to example 267, further comprising, subsequently to forming the sheet, and prior to temporarily flattening the sheet, fixedly attaching the sheet to the frame.

Example 269. The method according to any one of examples 263-268, wherein: (i) the frame is a first frame of multiple frames, fixedly attached to the sheet, (ii) flattening the sheet by constraining the frame comprises flattening the sheet by concurrently constraining the multiple frames while the multiple frames remain fixedly attached to the sheet, and/or (iii) allowing the frame to unflatten comprises allowing the multiple frames to unflatten.

Example 270. The method according to example 269, further comprising attaching the multiple frames in an arrangement across the sheet prior to flattening the sheet.

Example 271. The method according to example 270, further comprising, subsequently to allowing the multiple frames to unflatten, cutting the sheet to separate the multiple frames thereby forming multiple implants.

Example 272. The method according to example 269, wherein cutting the sheet comprises cutting the sheet while the multiple frames remain constrained.

Example 273. The method according to example 269, wherein forming the holes comprises forming the holes using an automated cutting device.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. A method for use in the manufacture of a medical implant, the method comprising:
forming a sandwich that includes:
a fabric layer,
a coating layer comprising a polymer configured to inhibit tissue growth thereon,
a binding layer sandwiched between the fabric layer and the coating layer, and
at least part of a frame sandwiched between the fabric layer and the binding layer;
heating and compressing the sandwich such that the binding layer binds the fabric layer to the coating layer; and
subsequently, forming holes through the sandwich.

2. The method according to claim 1, wherein forming the sandwich comprises sandwiching at least the part of the frame, along with the binding layer, between the fabric layer and the coating layer.

3. The method according to claim 1, wherein the coating layer consists essentially of the polymer.

4. The method according to claim 1, wherein the coating layer is nonporous.

5. The method according to claim 1, wherein the fabric layer includes a woven fabric.

6. The method according to claim 1, wherein the polymer includes an ultra-high-molecular-weight polyethylene (UHMWPE).

7. The method according to claim 1, wherein the binding layer comprises an acrylate.

8. The method according to claim 1, wherein the binding layer is 5-30 microns thick.

9. The method according to claim 1, wherein the part of the frame is 20-500 microns thick.

10. The method according to claim 1, wherein:
the coating layer defines a first face of the sandwich, and the sandwich has a second face opposite to the first face,
forming the sandwich comprises forming the sandwich to define a lip in which a lip-portion of the coating layer and a lip-portion of the binding layer extend together beyond an edge of the fabric layer, and
the method further comprises folding the lip around the edge of the fabric layer onto the second face, such that the lip-portion of the binding layer is sandwiched between the lip-portion of the coating layer and the second face.

11. The method according to claim 1, wherein the frame has a first side and an opposite second side, and wherein forming the sandwich comprises wrapping the fabric layer around the frame from the first side to the second side, such that at least part of the frame is sandwiched between (i) a first part of the fabric layer disposed against the first side of the frame, and (ii) a second part of the fabric layer disposed against the second side of the frame.

12. The method according to claim 1, wherein forming the sandwich comprises defining a lip in which a lip-portion of the coating layer and a lip-portion of the binding layer extend together beyond an edge of the fabric layer.

13. The method according to claim 1, further comprising, subsequently to heating and compressing the sandwich, flattening the sandwich, and maintaining the sandwich flat while forming the holes through the sandwich.

14. The method according to claim 1, wherein compressing the sandwich comprises compressing the sandwich at a pressure of 0.01-1 MPa.

15. The method according to claim 1, wherein the holes are hexagonal holes, and wherein forming the holes comprises forming the hexagonal holes.

16. A method for use in the manufacture of a medical implant, the method comprising:
forming a sandwich that includes:
a fabric layer,
a coating layer comprising a polymer configured to inhibit tissue growth thereon,
a binding layer sandwiched between the fabric layer and the coating layer, and
at least part of a frame sandwiched between the fabric layer and the binding layer; and
heating and compressing the sandwich such that the binding layer binds the fabric layer to the coating layer, wherein heating and compressing the sandwich comprises heating and compressing the sandwich in a manner that defines a pocket between the fabric layer and the binding layer, with at least the part of the frame disposed in the pocket.

17. The method according to claim 16, wherein heating the sandwich comprises heating the sandwich between plates, at least one of the plates having a recess at a region of the pocket.

18. The method according to claim 16, wherein compressing the sandwich comprises compressing the sandwich between plates, at least one of the plates having a recess at a region of the pocket.

19. The method according to claim 16, wherein heating and compressing the sandwich comprises heating and compressing the sandwich in a manner that provides an opening into the pocket where the fabric layer is unbound from the binding layer.

20. The method according to claim 16, wherein the medical implant is shaped to define a leaflet-augmentation patch, and the method further comprises attaching the leaflet-augmentation patch to a leaflet of a valve of a heart of a subject.
